(19) United States
(12) Patent Application Publication
(10) Pub. No.: US 2012/0093684 A1
(43) Pub. Date: Apr. 19, 2012

(54) UV sterilization system

(76) Inventors: John T. Martin, Riverside, CA (US); Lucien Tournie, Rowland Heights, CA (US); Douglas Gene Lockie, Los Gatos, CA (US)

(21) Appl. No.: 13/374,143

(22) Filed: Dec. 12, 2011

Related U.S. Application Data
(63) Continuation-in-part of application No. 12/456,714, filed on Jun. 19, 2009.
Application No. 12/456,714 is a continuation-in-part of application No. PCT/US2010/001716, filed on Jun. 14, 2010.

(51) Int. Cl. A61L 2/10; C02F 1/32; H05B 37/02; H05H 1/24; H05H 1/02

(52) U.S. Cl. 422/4; 315/111.21; 315/291; 422/23; 210/748.1

(57) ABSTRACT

Methods and apparatus for providing a UV Sterilization System are disclosed. In one embodiment, the present invention may be used as a fluorescent lamp ballast which is controlled using a non-resonant circuit that allows the ballast to lower to fifty percent the light output of the lamp while providing a corresponding fifty percent reduction in energy used.

### CROSS-REFERENCES TO PENDING PATENT APPLICATIONS & CLAIMS FOR PRIORITY

The Present patent application is a Continuation-in-Part patent application, and is related to:

Pending Parent patent application Ser. No. 12/456,714, filed on 19 Jun. 2009; and
Pending PCT International Patent Application No. PCT/US2010/001716, filed on 14 Jun. 2010.

The Applicants hereby claim the benefit of priority under Title 35 of the United States Code of Laws, Sections 119 and/or 120, for any and all subject matter which is commonly disclosed in the Present CIP patent application, and in either of the Parent and the PCT International Applications identified above.

The Applicants also incorporate all subject matter disclosed in the Parent and PCT International Applications identified above.

### FIELD OF THE INVENTION

One embodiment of the present invention pertains to methods and apparatus for providing fluorescent lighting. More particularly, one embodiment of the invention comprises a method for stimulating and maintaining the efficient operation of a fluorescent tube. Another embodiment comprises an improved method for sterilizing water.

### FEDERALLY SPONSORED RESEARCH OR DEVELOPMENT

None.

### BACKGROUND OF THE INVENTION

### Introduction

### The Fluorescent Lamp

Over 500 million fluorescent lamps are sold in the United States every year. Sales of “fluorescent lumiline lamps” commenced in 1938, when four different sizes of tubes were introduced to the market. During the following year, General Electric and Westinghouse publicized the new lights through exhibitions at the New York

World's Fair and at the Golden Gate Exposition in San Francisco. Fluorescent lighting systems spread rapidly during World War II, as wartime manufacturing intensified lighting demand. By 1951, more light was produced in the United States by fluorescent lamps than by incandescent lamps.

### How a Fluorescent Lamp Works

FIG. 1 depicts a generalized version of a conventional fluorescent lamp, which comprises a sealed glass tube filled with a gas that is maintained at very low pressure. When the gas is excited by applying an electrical current across the ends of the tube, particles generated by the excited gas strike the coating on the inside of the tube, and the coating emits visible light. (See GE Lighting, How It Works and Westinghouse Light Bulbs websites.)

A generalized pictorial view of a fluorescent lamp is depicted in FIG. 2. The fluorescent lamp is usually filled with a gas containing low pressure mercury vapor and argon, xenon, neon, or krypton. The pressure inside the lamp is around 0.3% of atmospheric pressure. The inner surface of the bulb is coated with a fluorescent (and often slightly phosphorescent) coating made of varying blends of metallic and rare-earth phosphor salts. The tube has two electrical terminals, a cathode and an anode. The cathode is typically made of coiled tungsten. This coil is coated with a mixture of barium, strontium and calcium oxides (chosen to have a relatively low thermionic emission temperature). When the light is turned on, the electric power heats up the cathode, and it begins to emit electrons into the lamp enclosure. The mercury atoms in the fluorescent tube must be ionized before the arc can “strike” within the tube. The electrons emitted from the cathode collide with and ionize noble gas atoms in the bulb surrounding the filament, and form a plasma by a process of impact ionization. The ultraviolet light is absorbed by the bulb's fluorescent coating, which re-radiates the energy at longer wavelengths to emit visible light. (See Wikipedia.)

A conventional incandescent light is shown in FIG. 3. An electrical current flows through a metal filament in an evacuated glass bulb. The electricity heats the wire filament, which produces a glow of visible light. A conventional incandescent light bulb is “electrically stable,” meaning that when the bulb is turned on, current flows through the filament at a relatively steady rate, and light is produced until the bulb is turned off. A fluorescent tube, by itself, is “electrically unstable.” When power is applied to an uncontrolled fluorescent light, more and more power flows into the lamp, and, eventually, the lamp burns up and is destroyed. This unfortunate result is due to an electric characteristic of the fluorescent lamp, which is based on the electrical property called “resistance.” In general, resistance is a characteristic of a substance to carry or convey a flow of electricity. Metals, like copper, gold and silver, are the best conductors of electricity, and have relatively low resistance. Insulators, like glass or plastics, do not allow electricity to pass, and, in general, have a relatively high resistance.

In a conventional incandescent light bulb, the resistance of a heated filament is relatively constant. In other words, once power is applied to a conventional light bulb, the filament heats up, and the amount of electricity that flows through the bulb remains about the same until the power is switched off.

In a conventional fluorescent lamp, after the power is initially supplied to the electrodes of the fluorescent lamp, the gas inside the tube is excited, and its electrical resistance begins to fall. More electricity flows into the lamp when the resistance drops, and the cycle continues unabated until so much current flows into the lamp, that the lamp is destroyed by excessive heat.

### Controlling the Fluorescent Lamp: The Ballast

The operation of conventional fluorescent lamps may be controlled by using an external device, called a “ballast,” which limits and regulates the current flow through the tube. The ballast may be a simple electrical component called a “resistor,” which limits the flow of energy into the lamp. A more prevalent form of ballast employs another electrical component called an “inductor,” which generally comprises a coil of wire wrapped around a metal core. Many different circuits have been used to start and run conventional fluorescent lamps. The design of a conventional ballast is based on input power voltage, tube length and size, initial cost, long term cost and other factors. (See Wikipedia).

### Supplying Power to a Fluorescent Lamp

Conventional fluorescent lamps may be powered by a direct current (DC), which flows in a steady stream, and which does not vary with time. In DC powered fluorescent lamps, the ballast must be resistive, and consumes about as much power as the lamp. Current day fluorescent lamps are almost never powered by direct current. Instead, the vast majority of present day fluorescent lamps run on alternating current (AC), which rises and falls in a regular cycle. FIGS. 4 and 5 furnish two graphs that compare direct current and alternating current.

More recent “electronic” ballasts utilize transistors or other semiconductor components to convert household voltage (120VAC) into high-frequency alternating current.

Beginning in the 1990's, a new type of ballast was introduced to the market. “High frequency” ballasts use high frequency voltage to excite the mercury within the lamp. These newer electronic ballasts convert the 60 Hertz household alternating current to a high frequency signal that can exceed 100 kHz. (See Wikipedia).

Present day conventional ballasts and fluorescent lamps are hampered by serious limitations. First, they consume substantial amounts of power. Second, they are not dimmable over a complete range of brightness. Third, every ballast must be especially configured for the particular fluorescent lamp with which it is to be used.

The development of an energy control device system that overcomes these limitations and that provides a substantial reduction in energy consumption would constitute a major technological advance, and would satisfy long felt needs and aspirations in the lighting industry, and would also satisfy pending and imminent regulatory demands.

### SUMMARY OF THE INVENTION

One embodiment of the present invention comprises a UV Sterilization System. One embodiment of the invention may be used to control the operation of a fluorescent lamp. One embodiment utilizes a circuit that enables light output dimming to one half of the maximum light output of the lamp, while simultaneously reducing energy consumption by fifty percent. Another embodiment of the invention may be used to kill microorganisms in water.

An appreciation of the other aims and objectives of the present invention and a more complete and comprehensive understanding of this invention may be obtained by studying the following description of a preferred embodiment, and by referring to the accompanying drawings.

### A BRIEF DESCRIPTION OF THE DRAWINGS

FIG. 1 provides an illustration of a spectrum of light, and shows a UV germicidal range of radiation.

FIG. 2 provides views of various embodiments of water disinfection devices.

FIG. 3 depicts a conventional fluorescent tube.

FIG. 4 shows how a conventional fluorescent tube operates.

FIG. 5 portrays the operation of a conventional incandescent light bulb.

FIGS. 6 and 7 are graphs that compare direct and alternating current waveforms.

FIG. 8 presents a generalized diagram which portrays one embodiment of the present invention.

FIG. 9 is a perspective view of a circuit board and components that may be used to implement one embodiment of the present invention.

FIGS. 10A, 10B & 10C are perspective views of components of the circuit boards shown in FIG. 9.

FIG. 11 provides a view of a spectrum of light, and shows the UV germicidal bands of radiation.

FIG. 12 shows one portion of one embodiment of the invention, which uses a generally cylindrical chamber with two bulb operating at half power.

FIG. 13 depicts a generally rectilinear bulb.

FIG. 14 illustrates an alternative embodiment of the rectilinear bulb shown in FIG. 13, which includes a reflective metal coating that acts as an electrical connection.

FIG. 15 portrays yet another alternative embodiment, which includes a cylindrical chamber for UV disinfection that includes four bulbs.

FIG. 16 supplies a view of another embodiment, which includes a rectilinear chamber that encloses round or rectangular bulbs.

FIGS. 17 and 18 furnish additional views of a rectilinear bulb with a reflective metal coating.

FIG. 19 illustrates the formation of ions at the electrodes within the bulb.

FIG. 20 offers a view of yet another embodiment, which comprises two spiral electrode elements.

FIG. 21 supplies an end view of the spiral electrodes shown in FIG. 20.

FIG. 22 is an enlarged view of one embodiment of the spiral electrodes.

FIG. 23 is an enlarged view of a group of protuberances formed on one side of a spiral electrode.

FIG. 24 is an enlarged view of one protuberance.

FIGS. 25 and 26 reveal embodiments of a UV generator with sharp points formed inside a glass cylinder.

FIG. 27 is a graph of one embodiment of the first electrical signal which is applied across the electrodes of the lamp.

FIG. 28 is a graph that shows one embodiment of the lamp current before an arc is initiated within the lamp.

FIG. 29 presents a graph of one embodiment of the first electrical signal after an arc is initiated within the lamp.

FIG. 30 supplies a view of one embodiment of current flowing through the lamp after an arc has been initiated.

FIG. 31 offers a graph of one embodiment of the combined first and second electrical signals employed by the present invention.

FIG. 32 provides a view of lamp current, sub-hyper.

FIG. 33 furnishes a graph which shows how one embodiment of the lamp voltage varies with time.

FIG. 34 supplies a graph which shows how one embodiment of the lamp current varies with time.

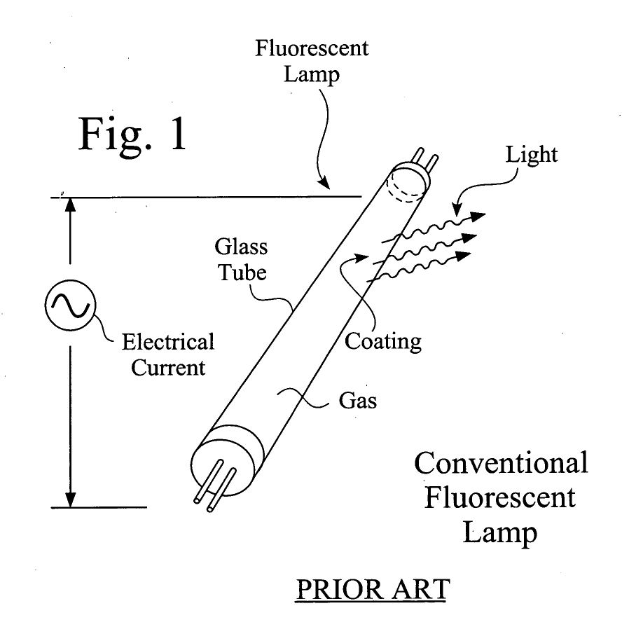

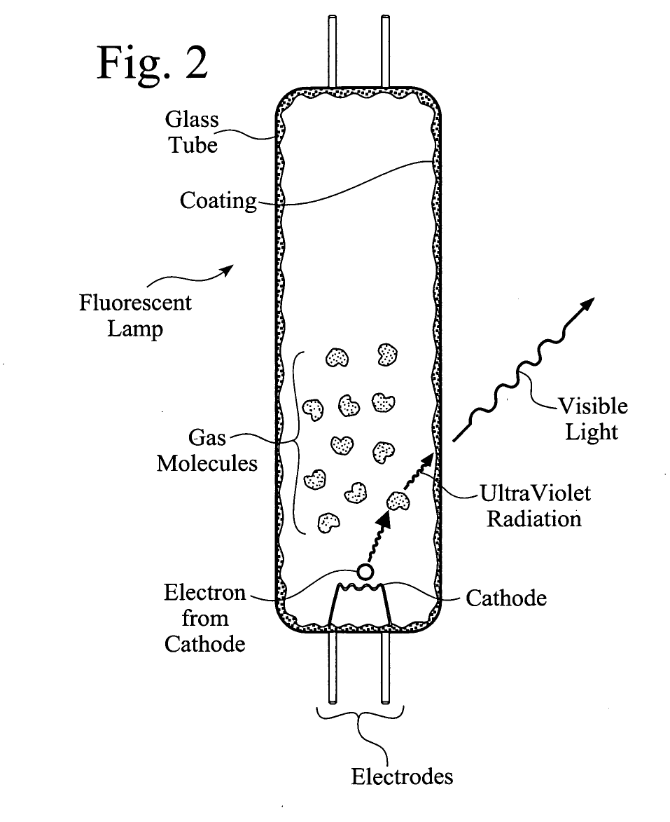

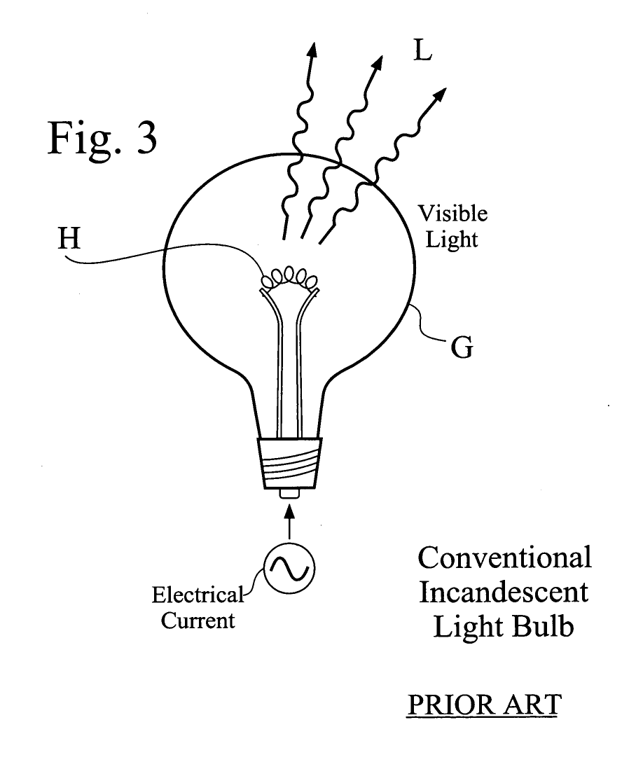

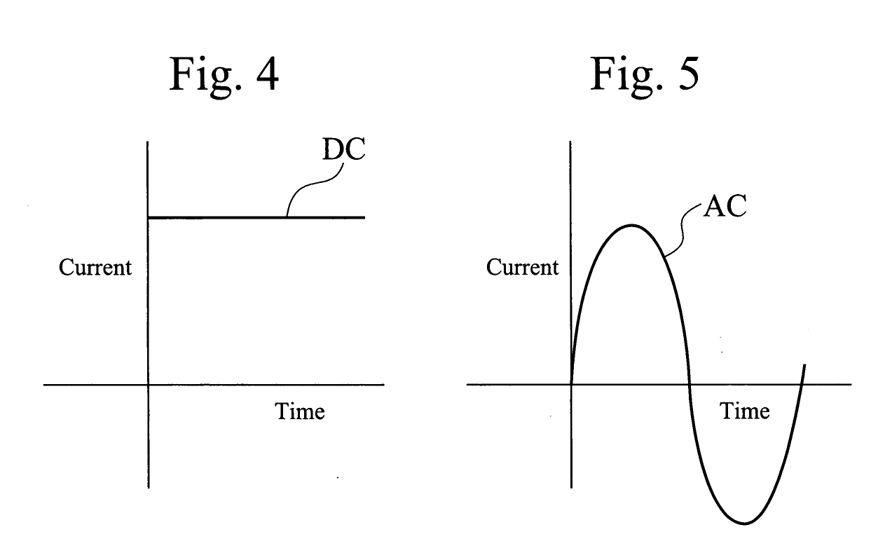

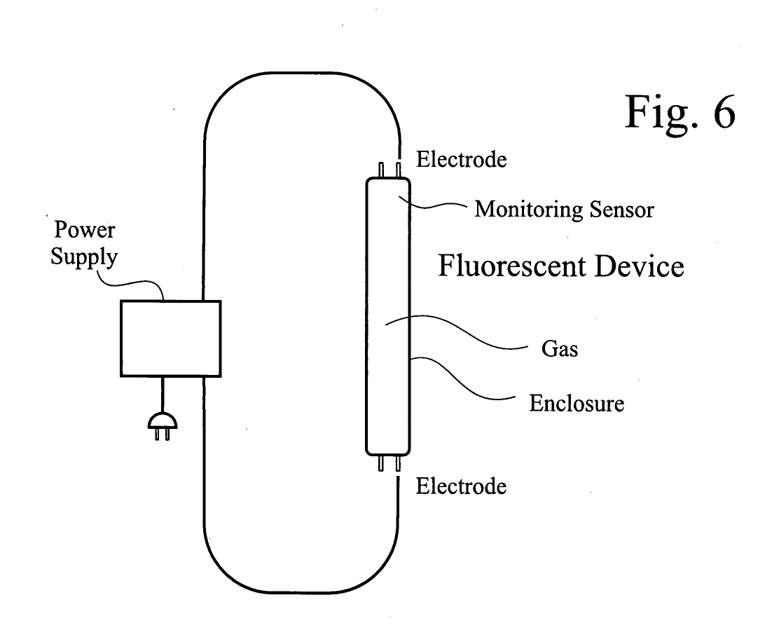

### A DETAILED DESCRIPTION OF PREFERRED & ALTERNATIVE EMBODIMENTS

### I. Fluorescent Lighting & UV Generation

The present invention comprises methods and apparatus for operating a highly efficient and dimmable fluorescent illumination device, or for generating ultraviolet radiation for disinfection and other purposes. In general, one embodiment of the invention provides for confining a plasma, and then stimulating the plasma with electrical energy to form a conductive plasma channel. This plasma drives the production of visible light by stimulating a photoluminescent substance surrounding the plasma. The impedance of the plasma channel, which varies with time and with ambient conditions, is then measured, and then the electrical input which maintains the plasma channel is adjusted to optimize its electrical impedance to provide efficient illumination. In another optional step, the polarity of the input waveform to the lamp is periodically reversed to eliminate any artifacts.

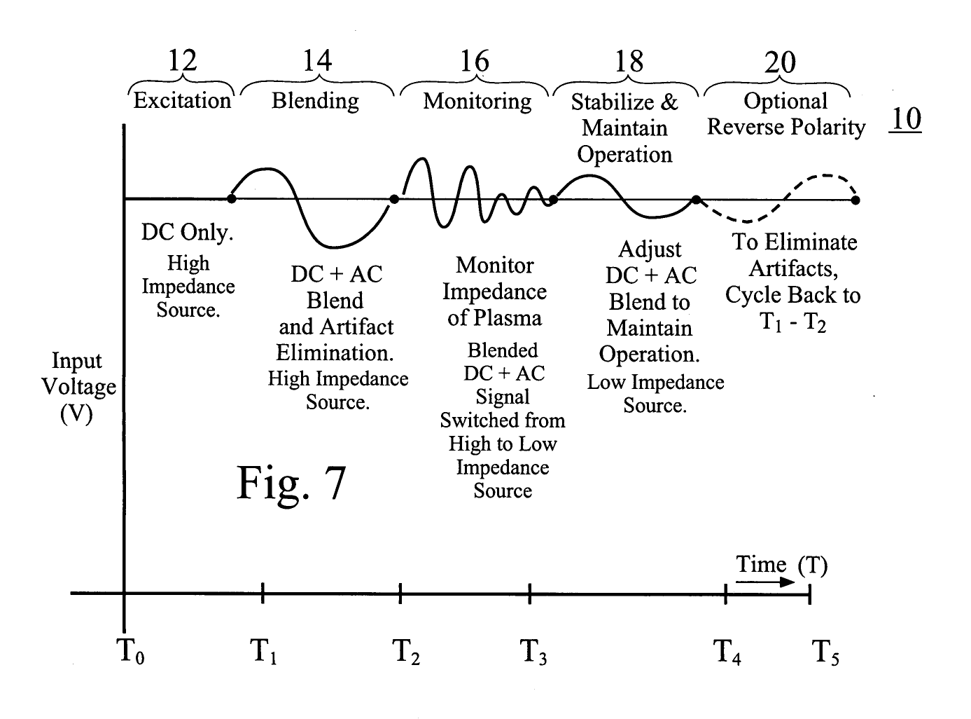

FIG. 6 reveals a fluorescent device connected to a power supply. The fluorescent device comprises a sealed enclosure which contains a number of molecules of gas which are capable of being ionized. In one embodiment of the invention, the enclosure is an optically transmissive substance, such as a cylindrical glass tube. In another embodiment of the invention, the enclosure may be a compact fluorescent bulb, configured as a spiral. The interior surface of the enclosure is coated with a light emitting substance. In one embodiment of the invention, the light emitting substance is a fluorescent or phosphorescent coating, which produces light when stimulated by ultraviolet radiation. One or more electrodes are located at either end of the tube, and generally extend from the outside of the tube into the enclosure. The electrodes may be disposed as pins, or may be connected through an electronic circuit to a threaded conductive end portion which fits into a standard light socket.

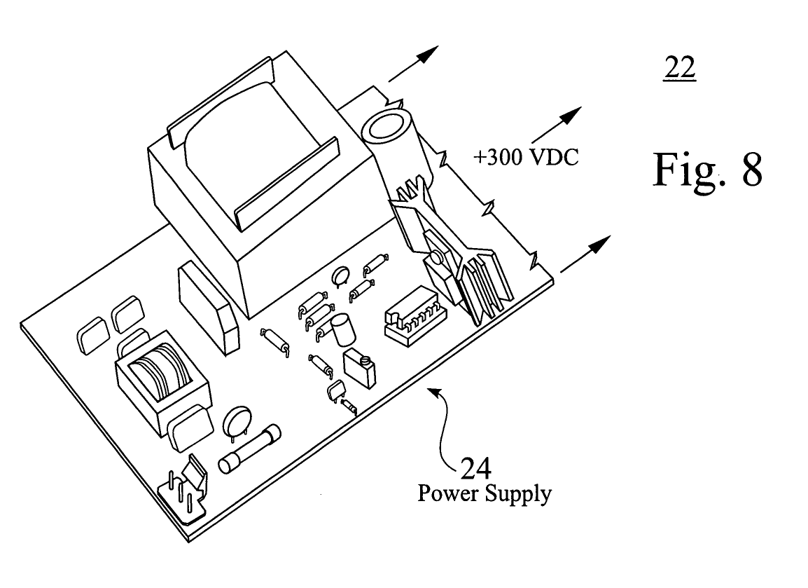

In accordance with one of the methods of the present invention, a first electrical signal is applied across the electrodes of the enclosure, as shown in FIG. 7. In one embodiment, this first electrical signal is a high voltage direct current voltage which excites the gas inside the enclosure, and which causes the gas to ionize, forming a plasma. This first electrical signal comes from a high impedance source. This first electrical signal is applied for a time T1-T0, which is labeled on the x-axis as the “Excitation Phase” of operation of the fluorescent device. The plasma is capable of conducting electricity between the electrodes through the center of the tube. The plasma generates ultra-violet radiation, which stimulates the phosphorescent coating of the inside of the tube, and produces visible light.

In the second phase of operation, which occurs between times T1 and T2, and which is labeled “Blending,” the first DC signal is blended with an AC signal.

In the third phase of operation, which occurs between times T2 and T3, and which is labeled “Monitoring,” the sensor inside the enclosure monitors the impedance of the plasma. When the conditions are correct, the source is switched from high impedance to low impedance.

In the fourth phase of operation, which occurs after time T3, and which is labeled “Stabilizing & Maintaining Operation,” an adjusted blend of DC and AC input signals are applied to the electrodes of the enclosure to maintain the optimal operation of the fluorescent device.

In an optional fifth phase of operation, which is labeled “Optional Polarity Reversal,” which may occur after time T4 the polarity of the waveform may be reversed to eliminate artifacts.

In one embodiment, the first signal is a relatively high voltage, constant direct current. In one embodiment, this first signal may range from 625 to 700 VDC. In one embodiment, the second signal is a mix of a constant direct current, and an alternating current. In one embodiment, the alternating current may range from 50 to 90 volts, and from 65,000 to 90,000 cycles per second. In an alternative embodiment, a series of direct current pulses may be substituted for the alternating current. In one embodiment, the second electrical signal ranges from 120 to 150 VDC.

In yet another embodiment of the invention, a radio may be attached to or installed inside the enclosure. This radio may be used to communicate to a remote transceiver to optimize the operation of the fluorescent device. A number of fluorescent devices, such as some or all of the bulbs on the floor of an office building, may use these radios to coordinate and control the operation of this group of fluorescent devices. In particular, these radios may be used for automatic dimming.

In one embodiment, the radio operates in the Wi-Fi frequency band, and can also be used to create a Wi-Fi hotspot for telecommunications.

In another embodiment, the power supply for the fluorescent device is built into the enclosure, and the invention operates without an external ballast. In another embodiment, the exterior surface of said enclosure also includes a partially mirrored surface to further enhance the optimization of the production of visible light from the light emitting substance on said interior surface of the enclosure.

In yet another embodiment of the present invention, priori knowledge of the characteristics of the enclosure are used to optimize the production of visible light from the fluorescent device.

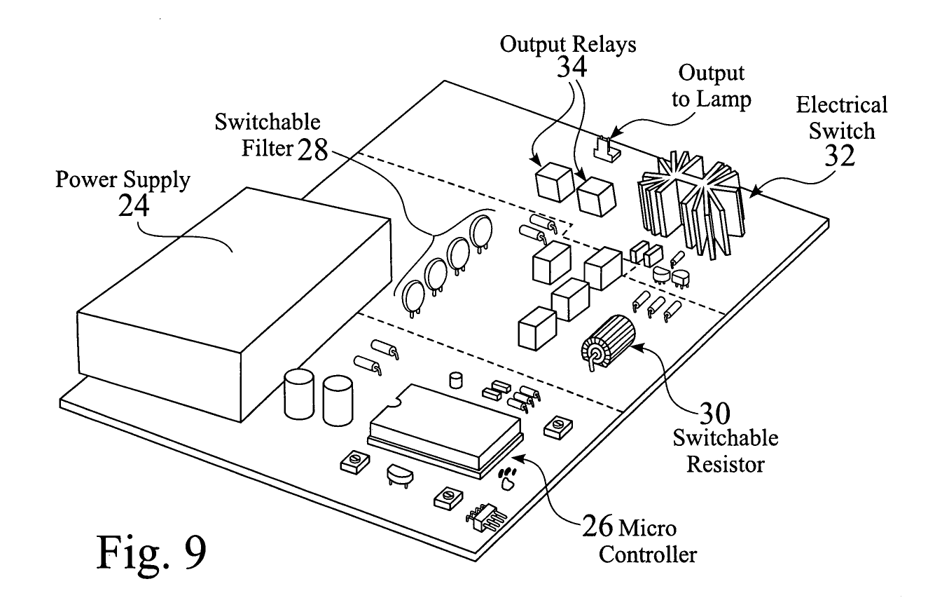

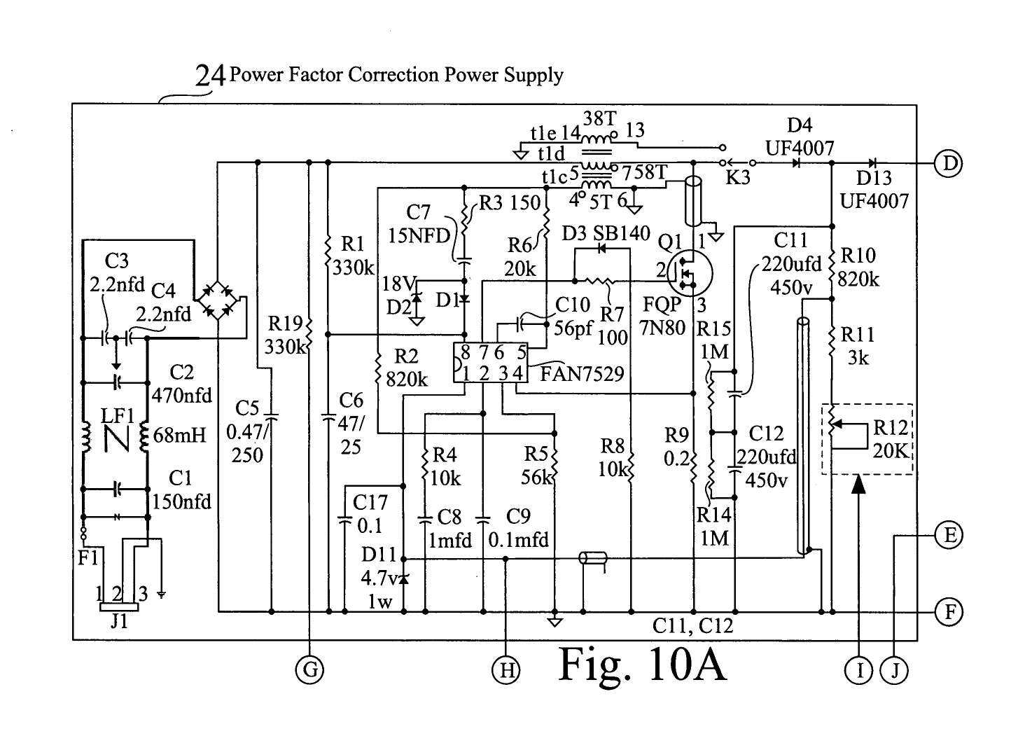

FIGS. 8 and 9 offer pictorial view of one embodiment of circuit boards which may be used to operate a fluorescent device in accordance with the present invention.

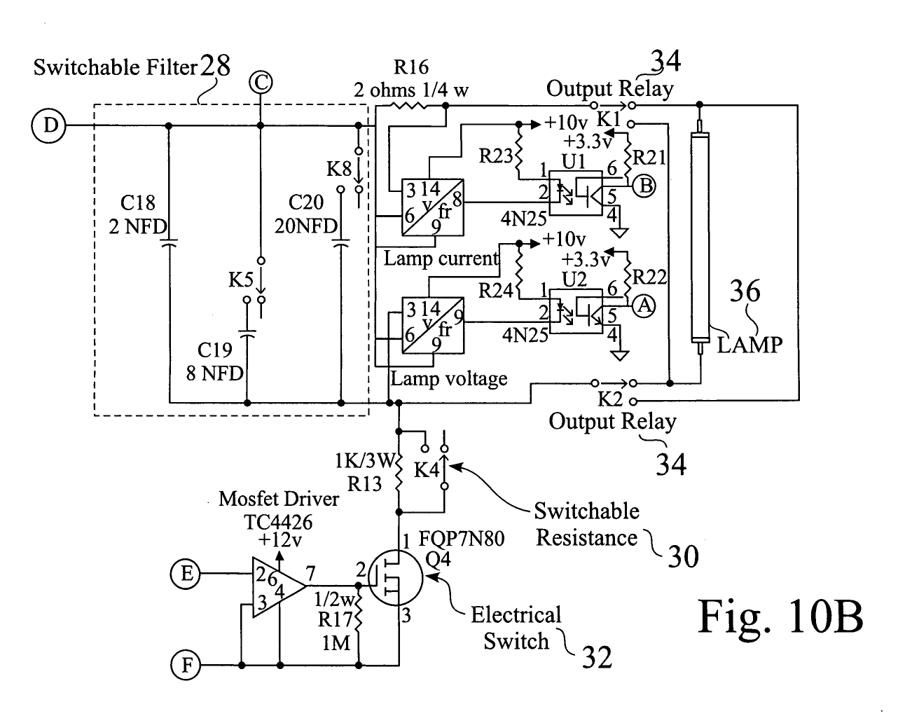

FIG. 10 provides a schematic diagram of the electronic components comprising the circuit boards shown in FIGS. 8 and 9.

The current through the lamp is monitored by the microcontroller when it interprets the frequency of the voltage pulse output from the optical isolator U-1 which is shown in FIG. 10. This frequency is generated by a “voltage to frequency” converter that samples the voltage developed across resistor R-16 which is shown in FIG. 10. The voltage across the lamp is monitored by the microcontroller when it interprets the frequency of the voltage pulse output from the optical isolator U-2 which is shown in FIG. 10. This frequency is generated by a “voltage to frequency” converter that samples the voltage developed between the common connection on output relay K-1 and the common connection on output relay K-2, which is shown in FIG. 10.

From these monitored quantities, the microcontroller calculates the lamp mean impedance characteristic. With this characteristic and the measured lamp mean current value, the microcontroller initiates the appropriate action by altering the voltage parameters applied to the lamp. For example, an F32T8 lamp operating at 100% illumination exhibits high efficiency when the measured mean current value is 0.180 ampere and the calculated mean impedance characteristic is 685 ohms. If the ambient temperature decreases, the lamp mean impedance characteristic will increase and the lamp efficiency will decrease. The microcontroller will react by adjusting the D.C. plus A.C. voltage amplitude and blend, applied to the lamp, to maintain 0.180 amperes and manage the impedance back to 685 ohms. High efficiency operation is restored.

The ballast microcontroller is also capable of dimming the light output. For example, the F32T8 lamp operates at high efficiency, at an illumination level of 37.5%, if the lamp means current value is 0.06 ampere and the calculated mean impedance characteristic is 2500 ohms. This is accomplished and maintained, by the microcontroller adjusting the D.C. plus A.C. voltage amplitude and blend applied to the lamp.

### Ballast Circuitry

One embodiment of the invention includes a microcontroller and firmware combination, a power supply, an electrical switch, a switchable resistance, a switchable filter and one or more relays that are used to reverse the polarity of the input applied to the lamp. Each component is described below.

### Power Supply

In one embodiment, the present invention works in combination with a power supply that converts incoming power line voltage, typically 110 VAC RMS, to an adjustable 100 VDC to 700 VDC at 100 watts. The power supply accomplishes this conversion from alternating to direct current while always making itself appearing as a resistive load to the incoming power line. This ability is called power factor correction (PFC). The circuit which accomplishes this task is readily available from a range of manufacturers. In one embodiment of the invention, a Fairchild model FAN7529 is employed as the power supply. See application note AN-6026. the output voltage is controlled by the microcontroller.

### Electrical Switch

In one embodiment, the present invention also works in combination with a one input electrical switch, which is used to control power fed to the lamp. When turned on its resistance should be less 0.3 ohms. When turned off, the break down voltage of the switch must be greater than 800 Volts. The switch is controlled by applying a voltage level shift which is supplied to a third connection on the switch. One embodiment of the invention utilizes a model FQP7N80 made by Fairchild. The electrical switch must be fast, making the transitions between states in less than 200 nanoseconds (200×10⁻⁹ seconds).

### Microcontroller and Firmware

In one embodiment, the present invention is also used in combination with a microcontroller and firmware. The microcontroller converts analog to digital and digital to analog, and must operate fast enough to perform calculations and run the ballast circuitry. A clock frequency of 20 MegaHertz is recommended. The recommended microcontroller family for one embodiment is the Zilog Z8 encore line.

The firmware monitors the amount of voltage and current that crosses and runs through the fluorescent lamp. The firmware must be capable of receiving an energy level instruction from an operator using the standard lighting industry communication protocols. The microcontroller may also be used to analyze the fluorescent lamp's condition, communicate the lamp type, and communicate that condition to the operator.

### Switchable Filter

In one embodiment, the present invention is also used in combination with a switchable filter. The filter resides in series between the power supply and the electrical switch, and it parallels the lamp and works with the switch to control the makeup (the alternating and direct current levels) of energy that powers the lamp. The fluorescent lamp performs best when the energy fed to lamp is filtered to be a mixture of 96-98% DC current and 2-4% AC current. The filter is switchable because its characteristics change depending on the level to which the lamp is being driven.

### Resistive Switch

In one embodiment, the present invention includes a switchable resistance. The switchable resistance resides in series with the power supply. The resistance is used to limit energy transfer to the lamp during the excitation and blending phases of operation. This occurs between T0 and T2, as shown in FIG. 7. Once past the blending phase and into the monitoring phase of operation, the resistance is switched out of the circuit. It is no longer used, unless it is needed to return to the blending phase for artifact elimination.

### Output Relay

In one embodiment, the present invention includes an output relay. The output relay resides between the switchable filter and the lamp. The relay is used to reverse the polarity of the electrical energy driving the lamp. Polarity reversal is utilized during the excitation phase of operation, T0 to T1, as shown in FIG. 7, and during the blending phase of operation, T1 to T2, as shown in FIG. 7.

### II. UV Sterilization

One of the most important methods that is currently employed to produce potable water employs sterilization by ultraviolet light. This process is referred to as Ultraviolet Germicidal Irradiation, or “UVGI.” This method may also be used to purify air, food, work surfaces, tools or other items that must be free from germicidal contamination. This method may also be used to destroy toxic chemicals. See Wikipedia website article, Ultraviolet Germicidal Irradiation.

Microorganisms such as bacteria and viruses, and larger organisms such as yeasts, fungi, protozoa, nematodes, molds and algae are present in virtually all untreated sources of water. When exposed to a particular frequencies of ultraviolet radiation, these microorganisms are destroyed. The ultraviolet light damages DNA or RNA within these germs, disrupts their ability to reproduce and prevents them from reproducing.

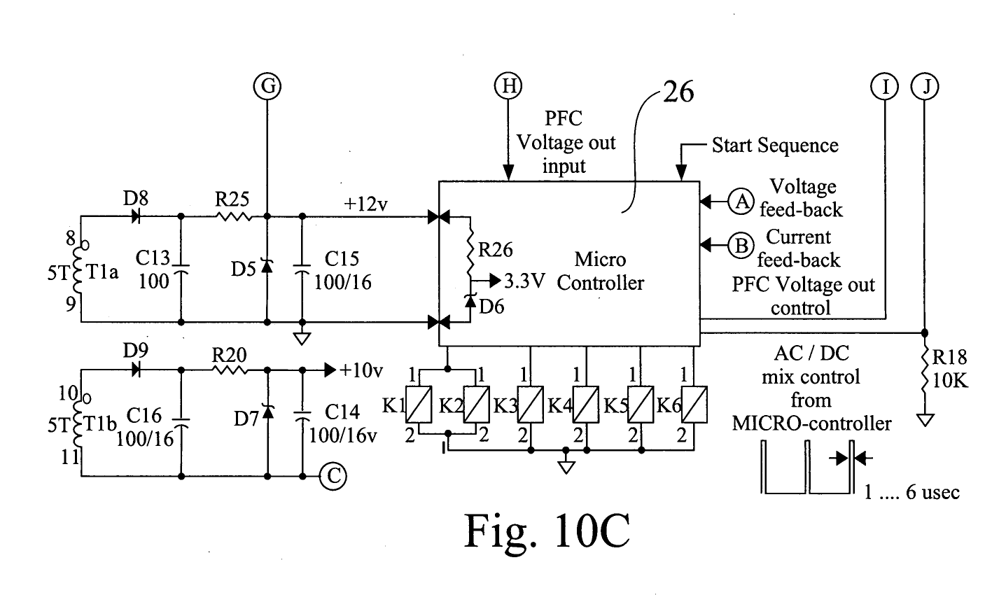

Ultraviolet light (UV) is a form of light which is invisible to human sight. UV light has a shorter wavelength than visible light. FIG. 11 shows a spectrum 37 that ranges from infrared up to x-ray radiation. UV is characterized by four segments or ranges, including UV-A, UV-B, UV-C and UV-Vacuum. The UV-C range is used for sterilization. The specific wavelength range which kills waterborne organisms is called the “germicidal spectrum,’ and lies in the 240-280 nanometer (nm) band. The frequency which most lethal is 264 nm. See Emperoraquatics website.

A fluorescent lamp may be used to generate the ultraviolet light for disinfection. A flow of water is directed through a device that includes a lamp which illuminates the water as it passes through the device. This method may be used to treat drinking water, wastewater, an aquarium, a pond, lab equipment, foods and beverages, or in manufacturing procedures utilized in the semiconductor industry. The amount of UV light which is required to kill various pathogens is different for each organism. Table One provides levels of UV exposure which are effective for disinfecting various germs.

TABLE One
Ultraviolet dosage required to destroy greater than 99.9% of micro-organisms.
Measured in microwatt seconds per centimeter squared.

| BACTERIA | |
|---|---|
| Agrobacterium tumefaciens | 8500 |
| Bacillus anthracis | 8700 |
| Bacillus megaterium (vegatative) | 2500 |
| Bacillus subtills (vegatative) | 11000 |
| Clostridium Tetani | 22000 |
| Corynebacterium diptheria's | 6500 |
| Escherichia coli | 7000 |
| Legionella bozemanii | 3500 |
| Legionella dumoffil | 5500 |
| Legionella micdadel | 3100 |
| Legionella longbeachae | 2900 |
| Legionella pneumophilla (legionnaires disease) | 3800 |
| Leptospira intrrogans (Infectious Jaundice) | 6000 |
| Mycobacterium tuberculosis | 10000 |
| Neisseria catarrhalls | 8500 |
| Proteus vulgaris | 6600 |
| Pseudomonas seruginosa (laboratory strain) | 3900 |
| Pseudomonas aeruginosa (environmental strain) | 10500 |
| Rhodospirllum rubrum | 6200 |
| Salmonella enteritidis | 7800 |
| Salmonella paratyphi (enteric fever) | 6100 |
| Salmonella typhimunum | 15200 |
| Salmonella typhosa (typhoid fever) | 6000 |
| Sarcina Lutea | 26400 |
| Seratia marscesens | 6200 |
| Shigella dysenterai (dysentery) | 4200 |
| Shigella Flexneri (dysentery) | 3400 |
| Shigella sonnell | 7000 |
| Staphylococcus epidermidis | 5800 |
| Staphylococcus aureus | 7000 |
| Streptococcus faecalls | 10000 |
| Streptococcus hemolyicus | 5500 |
| Streptococcus lactis | 8800 |
| Viridans streptococci | 3800 |
| Vibrio cholerae | 6500 |
| YEAST | |
| Bakers yeast | 8800 |
| Brewers yeast | 6600 |
| Common yeast cake | 13200 |
| MOLD SPORES | |
| Penicillum digitatum (olive) | 8800 |
| Penicillum expensum (olive) | 22000 |
| PeniciHum roqueforti (green) | 26400 |
| ALGAE | |
| Chlorella vulgaris (algae) | 22000 |
| VIRUSES | |
| Bacteriophage (E. coli) | 6600 |
| Hepatitis virus | 8000 |
| Influenza virus | 6600 |
| Pollovirus (pllomyelitis) | 2100 |
| Rotavirus | 2400 |

See Excelwater website.

The effectiveness of the sterilization process is proportional to the energy delivered by the lamp, and the length of time of the exposure.

The total UV energy emitted from all sides of the UV lamp is expressed in Watts. Over time, a lamps intensity decreases, and as a result, the UV output gradually decreases. Consequently, lamps must be replaced periodically for optimum efficiency.

The total exposure of the liquid is measured in microWatt seconds per centimeter squared (microWatts/cm2). The exposure is a product of the energy produced by the lamp over a certain amount of time and within a given area. A short exposure at a high intensity UV and a long exposure at a low intensity UV produce the same number of micro-Watt seconds per centimeter squared. See buyuv website.

The amount of ultraviolet energy the UV lamp produces is also dependent upon the primary voltage output and the lamp wall temperature. The effect of temperature is that the lamp will be only about 22% efficient in generating bactericidal radiation at 56.6F.° (12 deg.C.°). In one embodiment, a high intensity UV lamps inside a high-transmission clear fused quartz jacket is used, so that an optimum temperature of 104 F.° (40 C.°) is maintained for 100% UV output. See buyuv website.

When micro-organisms are subjected to ultraviolet light, a constant fraction of the number present die in each time increment. The fraction of the initial number of micro-organisms present at a given time is called the survival ratio. The fraction killed is one minus the survival ratio. The mathematical expression of these facts is shown below in Equation One:

$$Survival Ratio = Nt \text{/} No = e \text{-} KIt \qquad Equation One$$

### Where:

No=The number initially present
Nt=The number surviving at time
t=The time of exposure
I=the intensity (more correctly, scalar irradiance of ultraviolet light impinging on the microorganisms)
K=A constant which depends upon the type of micro-organisms and wavelength of ultraviolet light.

Equation One indicates that for each given micro-organism and UV wavelength, the fraction killed depends upon the product of UV light intensity and exposure time. This product is known as the “dosage.” It is the single most important parameter for rating UV disinfection equipment. See aquatechnology website.

### Device Design

The basic design problem in any ultraviolet system to efficiently and reliably deliver the required dosage to micro-organisms suspended in the fluid. There are essentially two basic design concepts in current use to accomplish this task. One employs flow over a submerged bank of germicidal lamps with quartz sleeves. Fluid flows through Teflon® tubes surrounded by germicidal lamps in the other design concept.

Quartz Tube Systems with Shellside Flow

Most substances are not penetrated by short-wave ultraviolet rays. Water is one of the few liquids which allows a significant penetration. Quartz is one of the few solid materials that is virtually transparent to short-wave ultraviolet. It is used in the manufacture of germicidal lamps and as a material of construction in many commercial ultraviolet systems.

Quartz tubes tend to be brittle, fragile and difficult to seal. Complicated in-place cleaning systems must usually be installed to keep the quartz surface free from fouling materials.

In a conventional quartz ultraviolet disinfection unit, water flows over a bank of quartz sleeves similar to flow in the shell-side of a shell-and-tube heat exchanger. Inside each quartz sleeve is a germicidal lamp. A separate o-ring seal is made at the end of each quartz sleeve. The outer shell is usually constructed of either stainless steel andized aluminum, or polyvinyl chloride. Quartz UV systems are designed for either pressurized or gravity water flow.

Unless the quartz ultraviolet system is disinfecting an ultra-pure water source, the quartz sleeves readily foul with suspended and dissolved matter in the water. Therefore, it is necessary to employ a technique to remove the fouling matter on an almost continuous basis to preserve the high UV transmittance of quartz and the disinfection capability of the system.

Two, non-chemical, cleaning methods have been used with limited success. The first is a mechanical wiper system, in which a wiper periodically scrapes fouling deposits off of the outer surface of the quartz sleeves. For this technique to work effectively, very close tolerances are required on quartz sleeve outer diameter and alignment. Close tolerances are also required on the wiper system as well. These severe tolerance requirements add to manufacturing expense and tend to be difficult to achieve with large tube bundles.

### Teflon Tube Flow Systems

In the early 1970's, it was discovered that FEP Teflon was also an excellent transmitter of 254 nm ultraviolet light. Data obtained over fifteen years of continuous testing by DuPont indicated that Teflon was also very stable to solar ultraviolet (minimum 290 nm). Shorter term tests (five years) indicated that Teflon is virtually unaffected by the shorter germicidal wave length light. Teflon, an deal material to contain a wastewater during disinfection, has the following advantages:
1. It has a high transmission of 254 nm ultraviolet light—approximately 80 percent transmission with wall thicknesses used in disinfection systems.
2. Teflon is chemically inert. Teflon tubes are not attacked by substances present in water or wastewater.
3. It is non-wetting and has an extremely low-friction co-efficient. Teflon tubes are usually not fouled by substances present in water or wastewater. If fouling should occur, chemical cleaning can be used to easily remove deposits.
4. Teflon is an approved material by the U.S. Food and Drug Administration for use with food and beverages.
5. It is virtually unaffected by ultraviolet rays.

In the Teflon tube flow system, the fluid to be disinfected flows through Teflon tubes. To achieve large flow capacities, these tubes can be connected in parallel to large diameter headers. Systems of several million gallons per day capacity can then be built as a single unit.

Banks of germicidal lamps are placed in between the tubes so that each tube is exposed to ultraviolet light from alt sides. The lamps are mounted on a frame which can be slid out for easy lamp replacement.

Aluminum, which is an excellent reflector for ultraviolet, forms the outer casing. Unabsorbed ultraviolet, which strikes the enclosure walls, is mostly re-reflected and is eventually absorbed by the water. This design is extremely efficient in the utilization of ultraviolet energy emitted by the germicidal lamps.

Ultraviolet irradiation involves maintaining an optimal dose of radiant energy.

Manufacturers of ultraviolet irradiation equipment provide ratings related to the maximum water flow rates which may be attempted, above which the radiant dose will be inadequate.

While there will be some variations among different makes and models, most ultraviolet disinfection equipment of the type used for hemodialysis and other purified water systems are designed to provide a radiant dose of 30,000 microWatt-sec/cm2, which is well in excess of that needed for the destruction of most, but not all, types of water-born bacteria.

An inadequate dose of radiant energy may result if the mercury vapor lamps are not periodically replaced, if the water contains materials which absorb or prevent the light from reaching the bacteria, or if the quartz sleeve becomes coated.

For many commercial units, a continuous flow of water is required to prevent overheating of the ultraviolet lamps. If such overheating is allowed to occur, the wavelength of ultraviolet light emitted may change to a point at which it is no longer bactericidal and, when flow is resumed, initial volumes of effluent water may be bacterially contaminated. See aquatechnology website.

### UV Applications

### Well Water

Many rural homeowners who draw their water from private wells assume that their water is safe. Unless the water has been tested, however, there is no way to know whether it contains potentially harmful pathogens. A coliform count indicates that a well is contaminated. Faulty sewage or manure systems or field run-off can be sources of the contamination.

Many livestock producers wish to protect their animals from poor water quality and install water treatment systems that incorporate UV for disinfection.

### Surface Water

In many rural regions, homes and cottages draw their water directly from lakes or streams, which collect potentially harmful storm run-off. Add that many animals live in these lakes and streams, and the likelihood of microbial contamination in these supplies is high. Again, the water can be tested, and a coliform count will indicate whether the water should be disinfected.

### Public Water Supplies

Even people in communities served by municipally treated water are installing UV systems. Concerns over the health affects of chlorine have prompted many families to de-chlorinate their water. Some of these families use UV systems to disinfect their de-chlorinated water. Others install UV systems to back up the municipal treatment process.

### Commercial Water

Restaurants, hotels, resorts, and campgrounds must supply safe water to their guests. Many of these establishments now employ UV disinfection systems because they are simpler and easier to handle than chlorination systems.

As well, the sick and elderly are more susceptible to waterborne pathogens than are the young and healthy. Consequently, hospitals and nursing homes must keep their water free from microbial contamination. The medical industry also incorporates UV into essential processes such as dialysis.

### Process Water for Industry

Factories and laboratories with low water use but high quality requirements can take advantage of UV disinfection systems to treat their water. Some processes are unable to tolerate chlorine, and the food and beverage industry wants to eliminate the odour and taste of chlorine from their products.

### III. Preferred & Alternative UV Sterilization Embodiments of the Invention

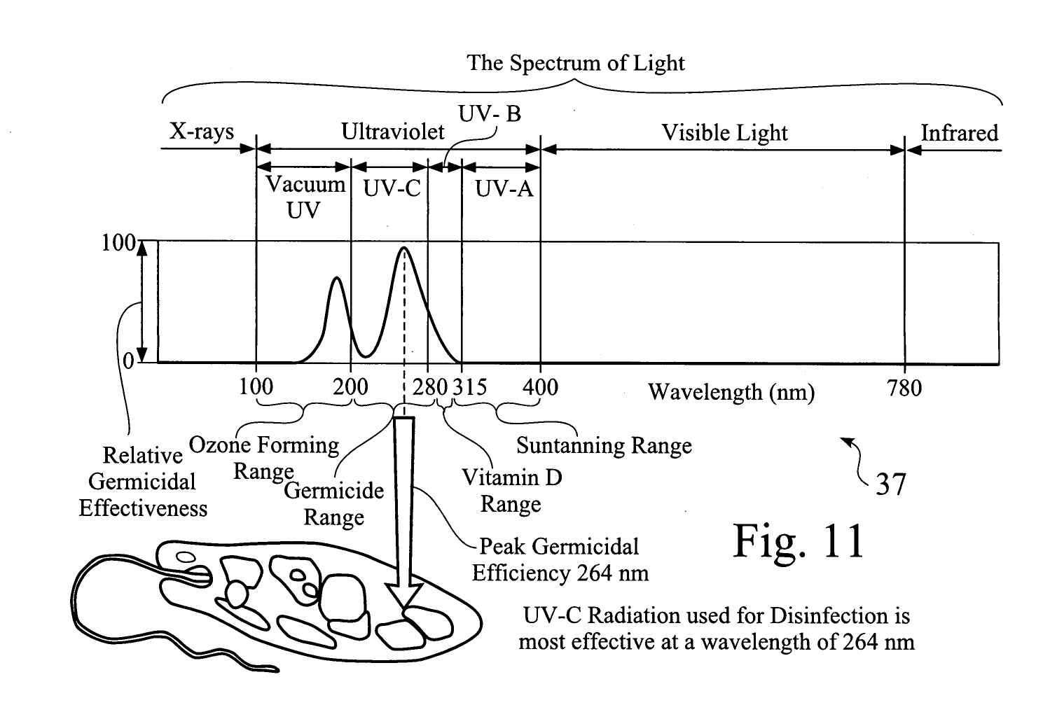

FIG. 12 provides a view of one embodiment 38 of the present invention. This embodiment includes a outer UV transparent quartz jacket 40 surrounded by a water 42. Two UV bulbs 44 and 46 reside in the center of the inside of the jacket 40, and are connected to a source of electrical power by first and second electrodes 48 and 50. The two bulbs 44 and 46 are configured to run at half power to extend their useful lifetime.

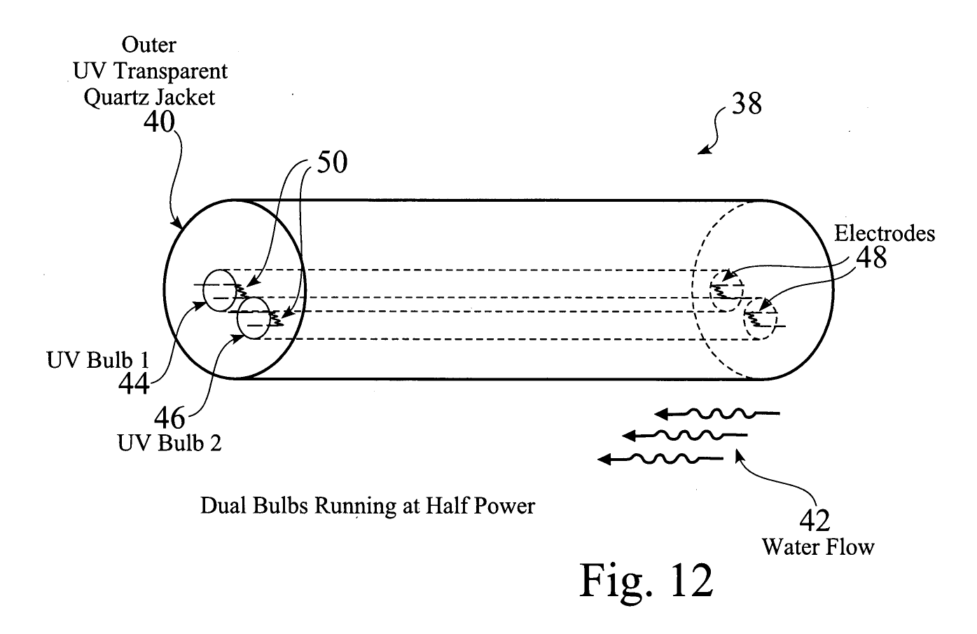

FIG. 13 offers a view of another embodiment of the invention, which includes a generally rectilinear enclosure 54. A power source 56 is connected to electrodes 48 and 50. A reflective layer such as aluminum, silver, copper or a combination of suitable metals is used both as a reflector to enhance the operation of the enclosure 54.

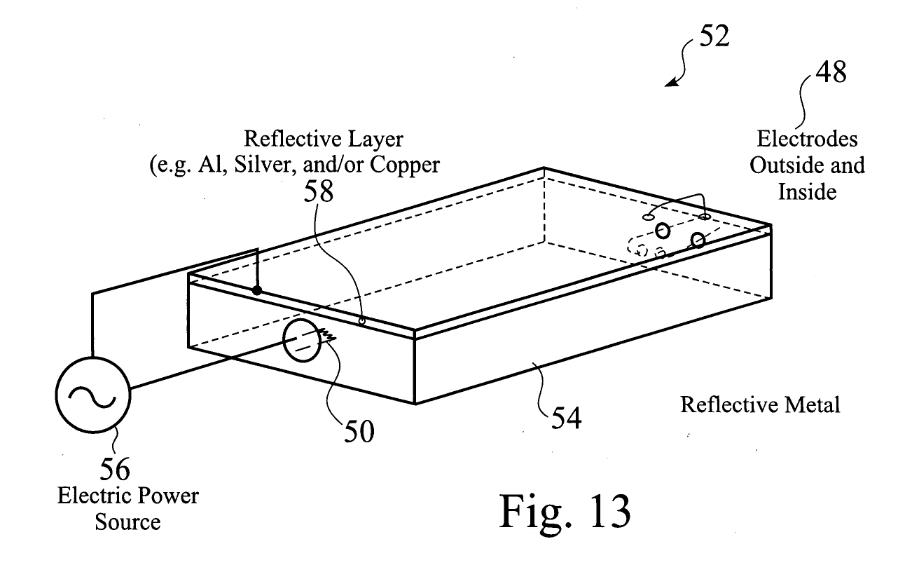

FIG. 14 supplies a view of another alternative embodiment 60 of the invention. A reflective metal coating 62 not only acts as a reflector, but also serves as an electrical connection for the bulb.

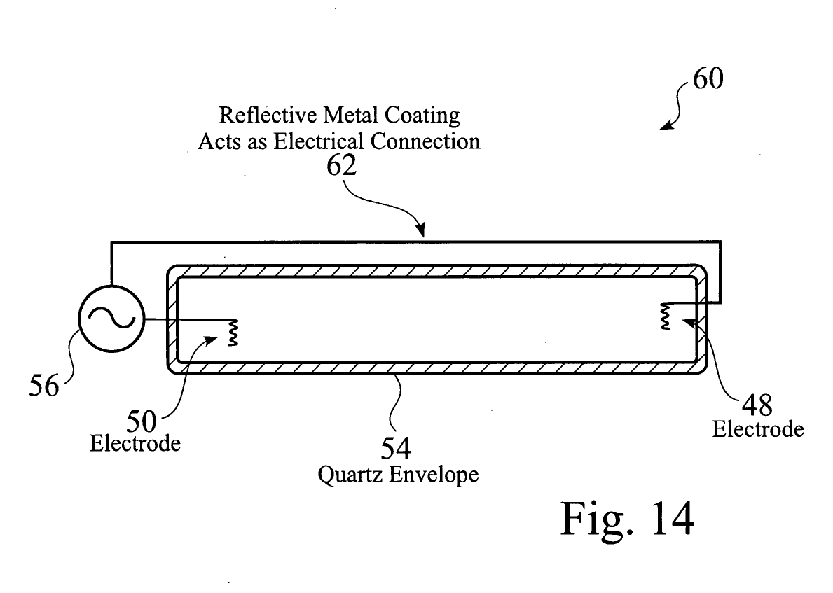

FIG. 15 depicts one embodiment of a water sterilizer 64 built in accordance with the present invention, which includes a water inlet pipe 66, an outlet pipe 68, a generally cylindrical chamber 70, and four bulbs 72 located within the chamber 70. This embodiment may be used to disinfect not only water, but also any other suitable liquid, air, gas, mixture, slurry or material that is capable of flowing through the chamber 70.

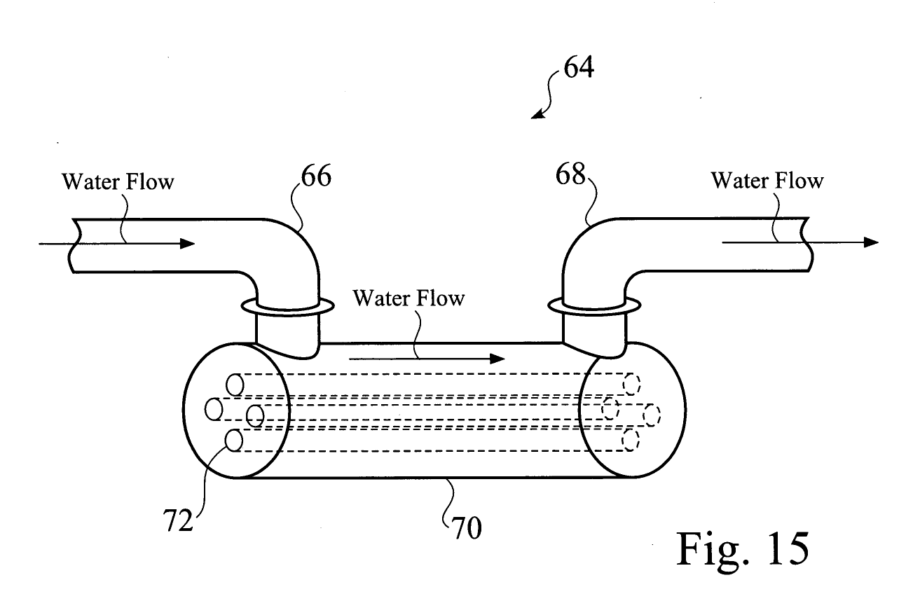

One variation 74 of the embodiment shown in FIG. 15 is presented in FIG. 16. A generally rectilinear chamber 76 encloses a number of round or rectangular UV tubes 78.

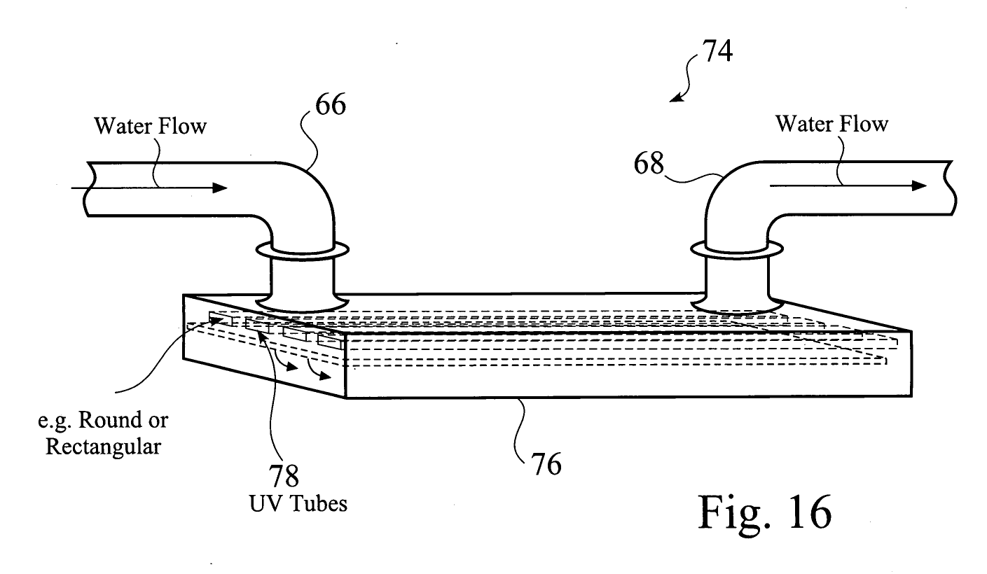

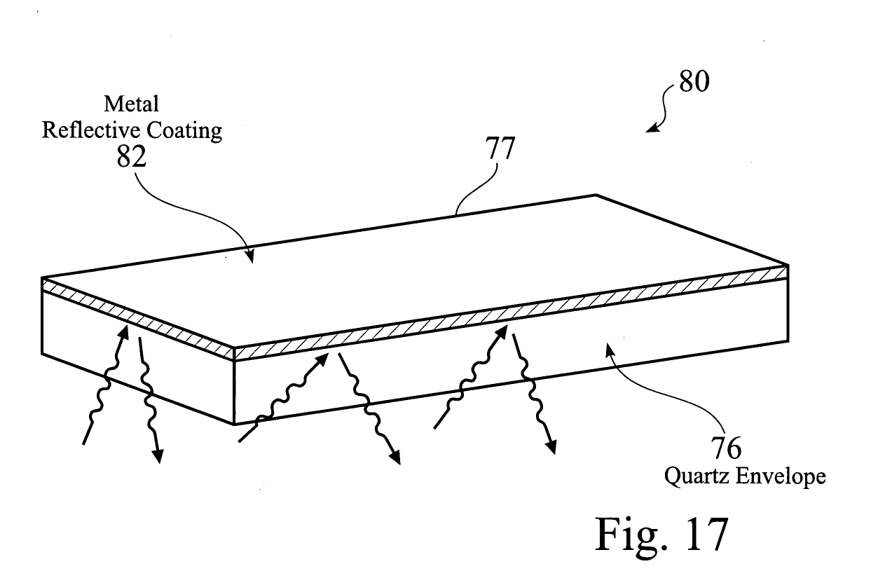

FIG. 17 furnishes a view of an alternative embodiment 80, which includes a rectilinear bulb 77 that includes a reflective metal coating 82 for enhancing the operation of the bulb. In general, the bulb may be configured in any shape or geometrical shape which is suitable for its specified purpose. FIG. 18 supplies a cross-sectional view of the rectilinear bulb depicted in FIG. 17.

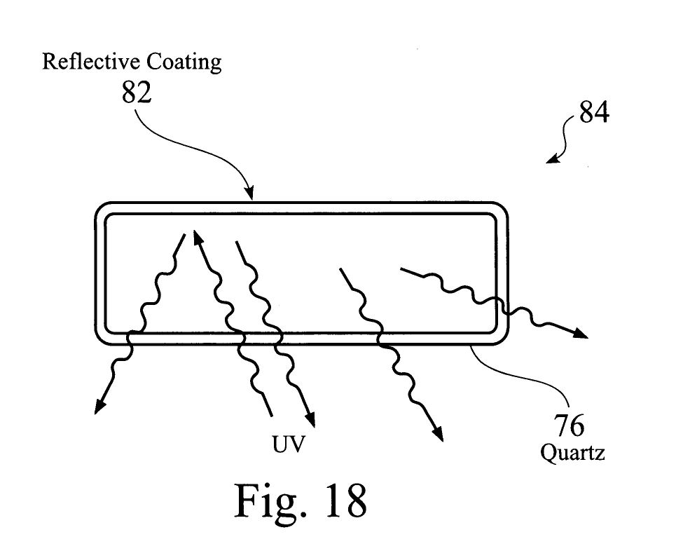

FIG. 19 presents a schematic view 86 of the formation of ions near the electrodes of the bulb. A quartz envelope 88 encloses first and second electrodes 48 and 50, which are connected to power supplies 56 and a switch 90. Ions 92 are formed in the gas surrounding the electrodes when the bulb is operating.

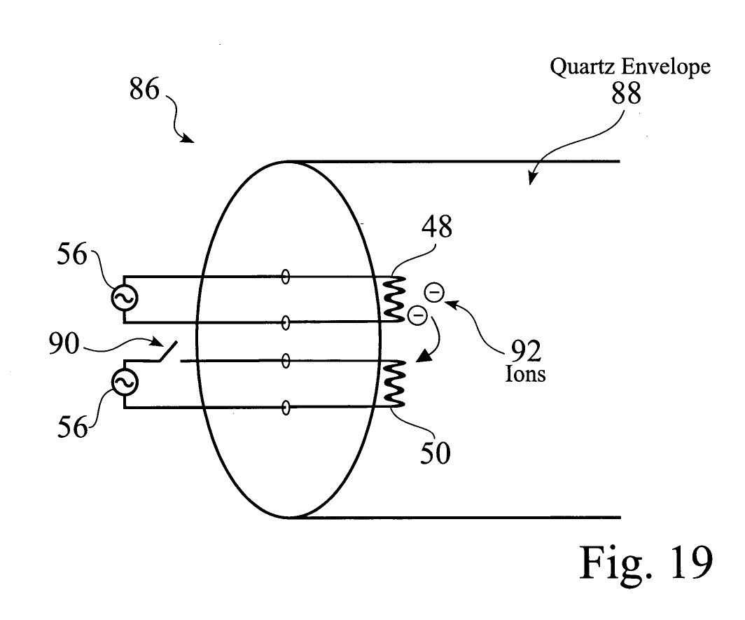

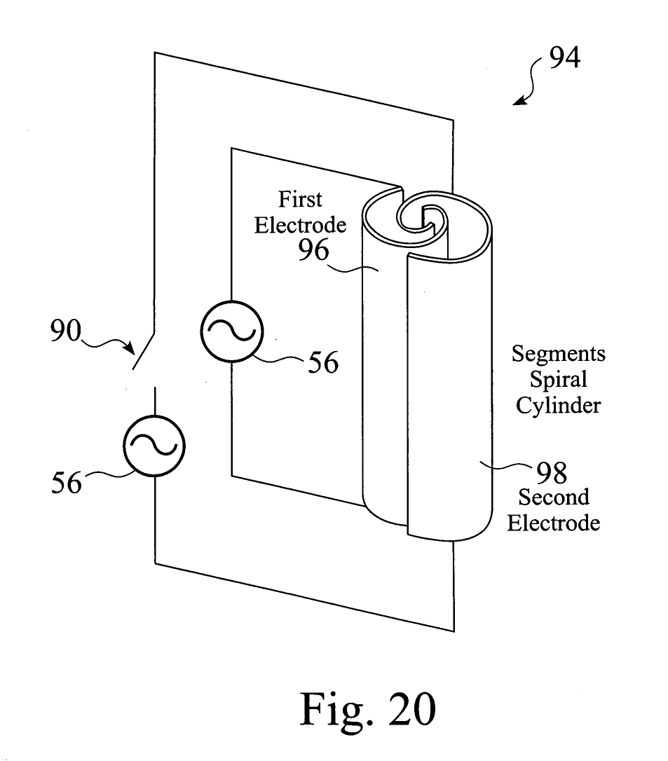

FIG. 20 offers a view of yet another embodiment 94 of the invention. Two generally cylindrical, spiral electrodes 96 and 98 are deployed in close proximity to enhance the operation of the bulb. FIG. 21 supplies an end view 100 of the spiral electrodes 96 and 98.

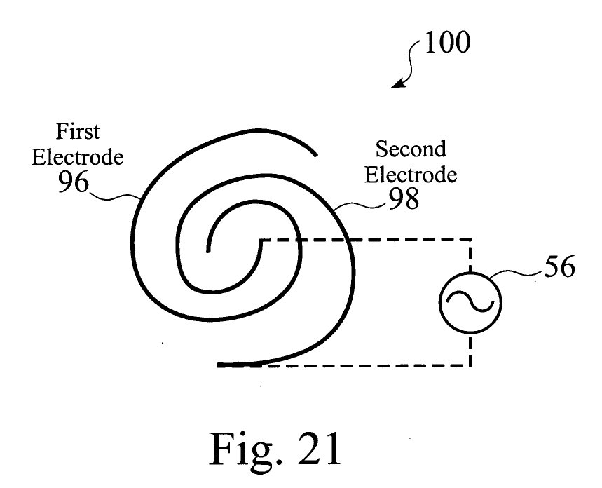

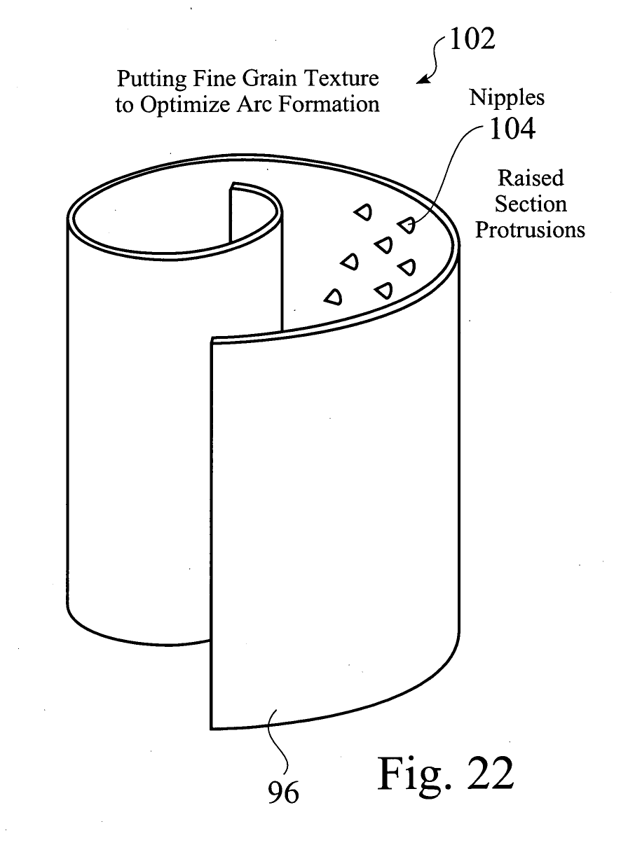

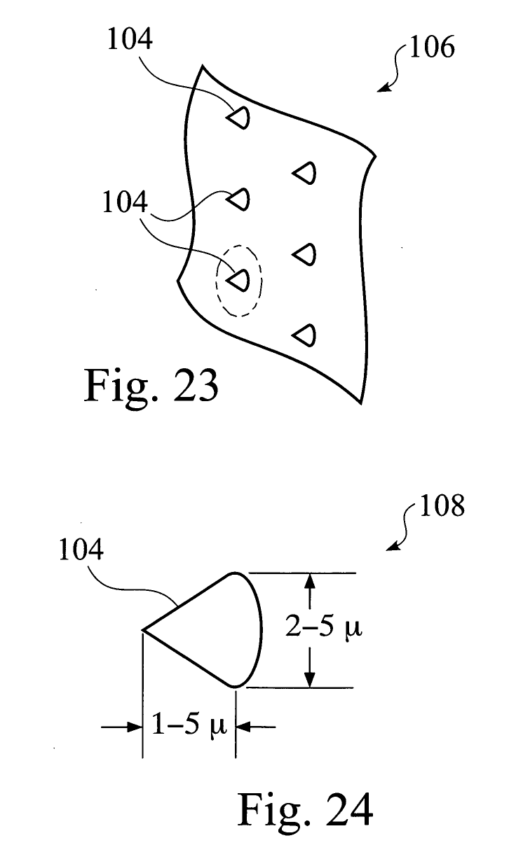

FIG. 22 provides a view 102 of an improved spiral electrode 96, which includes protuberances 104 formed on the inward facing surface. These protuberances 104 are bumps, points, extensions, swellings, ridges or any other suitable raised portion of an otherwise smooth surface which enhance the operation of the bulb. FIGS. 23 and 24 are enlarged views 106 and 108 of these protuberances 104. In one particular embodiment, these protuberances are formed in a cone shape which may range from one to five microns high, and from 2 to five microns wide at their base.

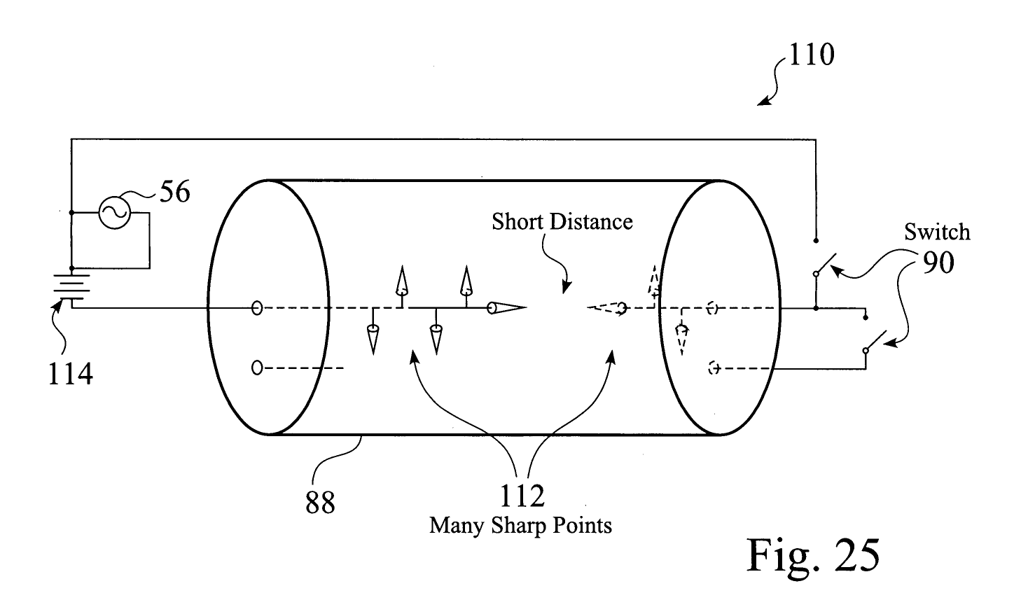

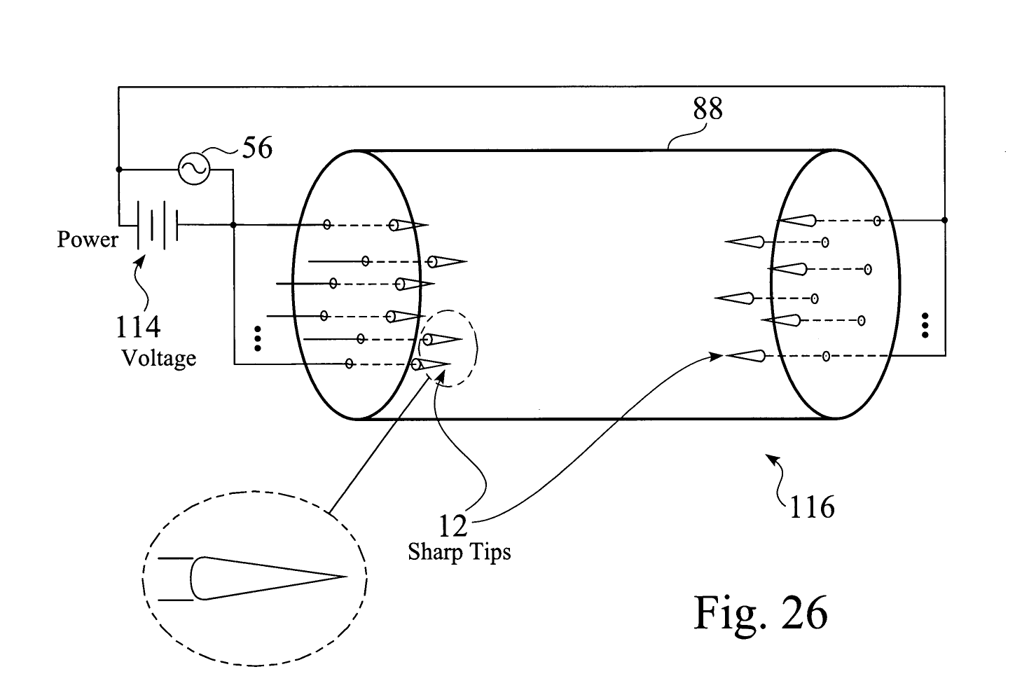

FIG. 25 depicts yet another alternative embodiment 110, comprising a cylindrical bulb that includes many sharp points 112 deployed at the at end of a needle or filament that is connected to an electrode. In one embodiment, the sharp points 112 are generally short elongated conductors which enhance the operation of the bulb. These sharp points concentrate the surrounding electrical field, and enhance the ability of the bulb to strike an arc. FIG. 26 supplies a view of an alternative embodiment 116 of a bulb with sharp points. This embodiment includes a battery 114, and has sharp points located at either end of the bulb.

### III. Alternative Embodiments of the Invention

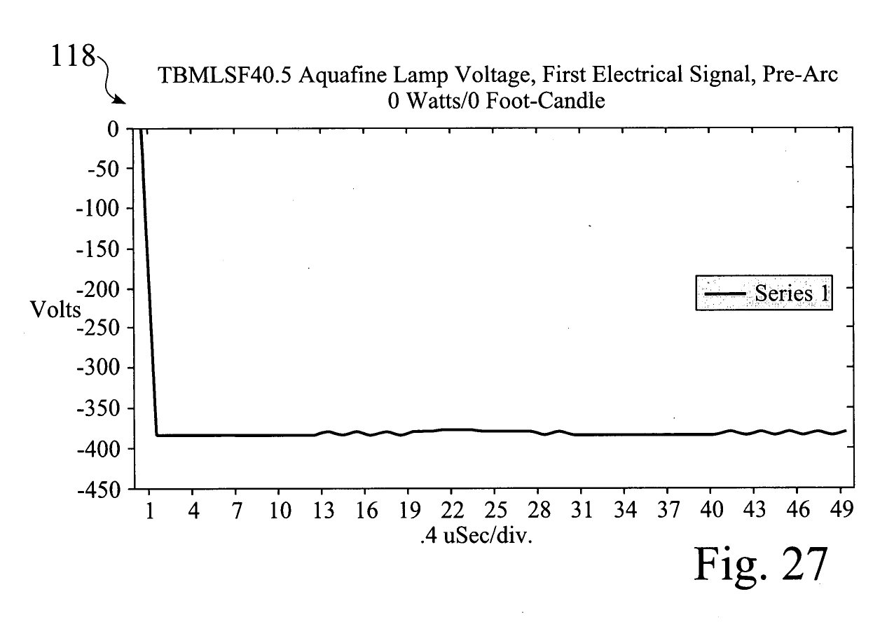

FIG. 27 is a graph of one embodiment of the first electrical signal which is applied across the electrodes of the lamp.

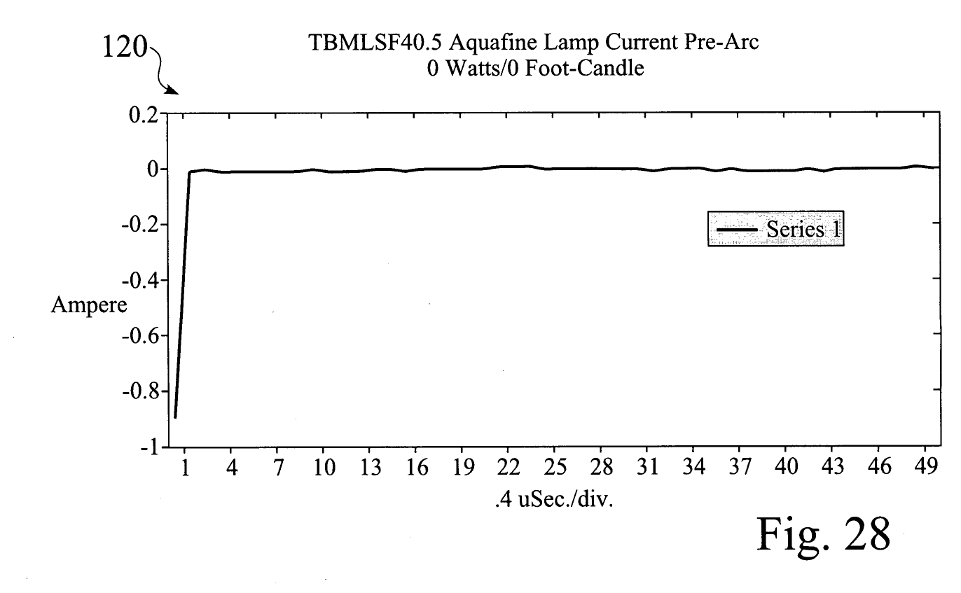

FIG. 28 is a graph that shows one embodiment of the lamp current before an arc is initiated within the lamp.

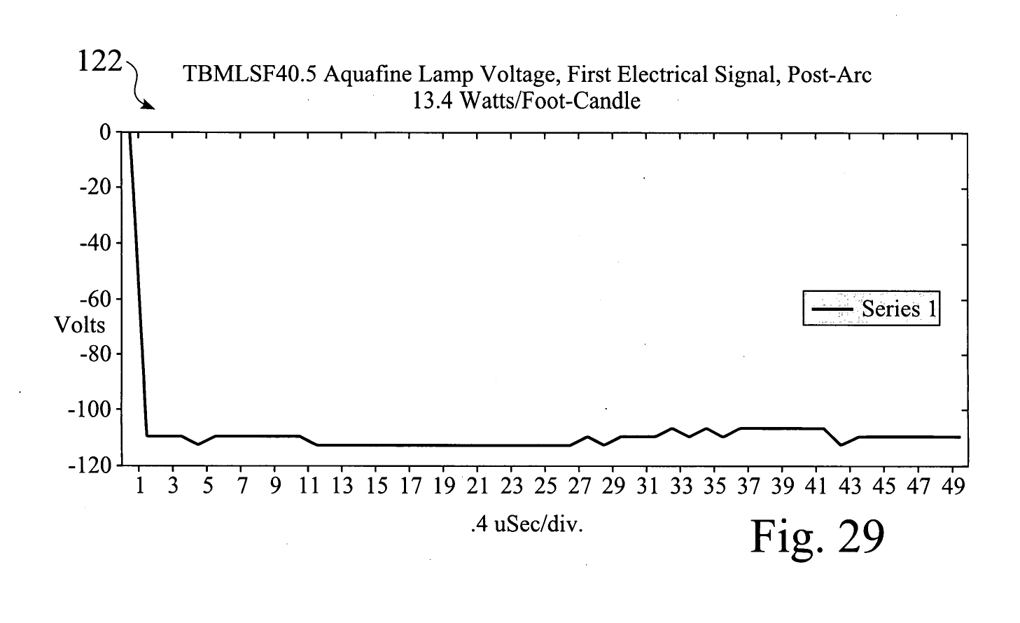

FIG. 29 presents a graph of one embodiment of the first electrical signal after an arc is initiated within the lamp.

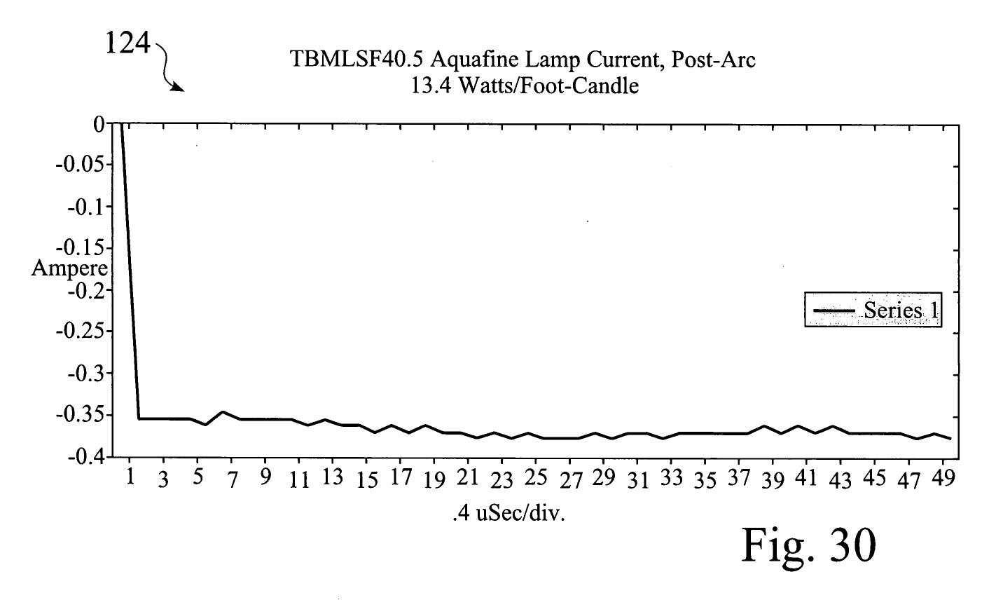

FIG. 30 supplies a view of one embodiment of current flowing through the lamp after an arc has been initiated.

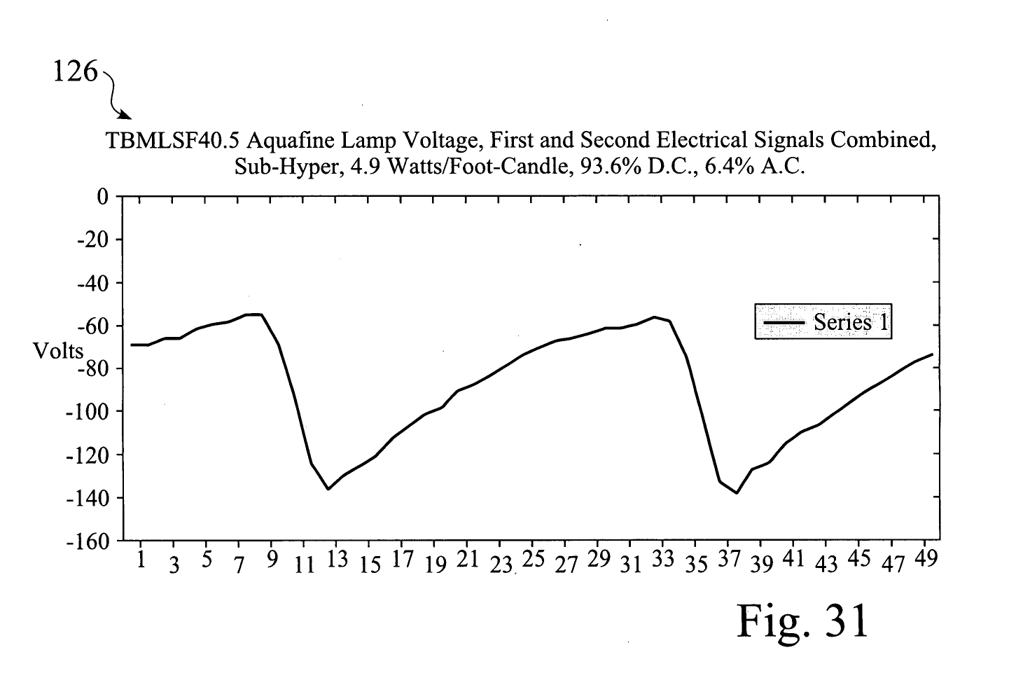

FIG. 31 offers a graph of one embodiment of the combined first and second electrical signals employed by the present invention.

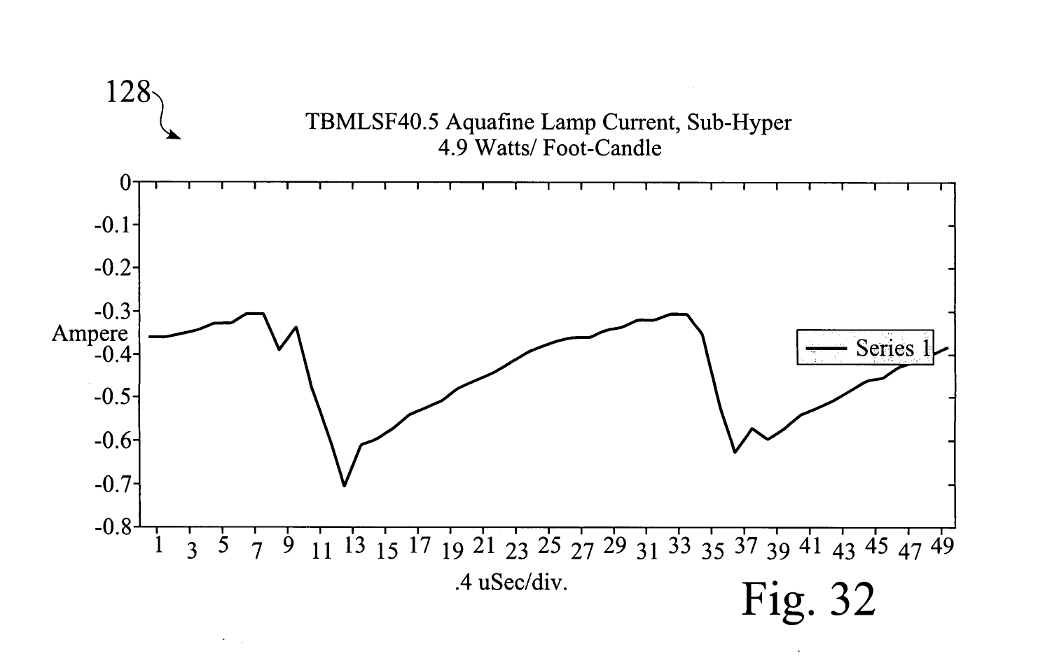

FIG. 32 provides a view of lamp current, sub-hyper.

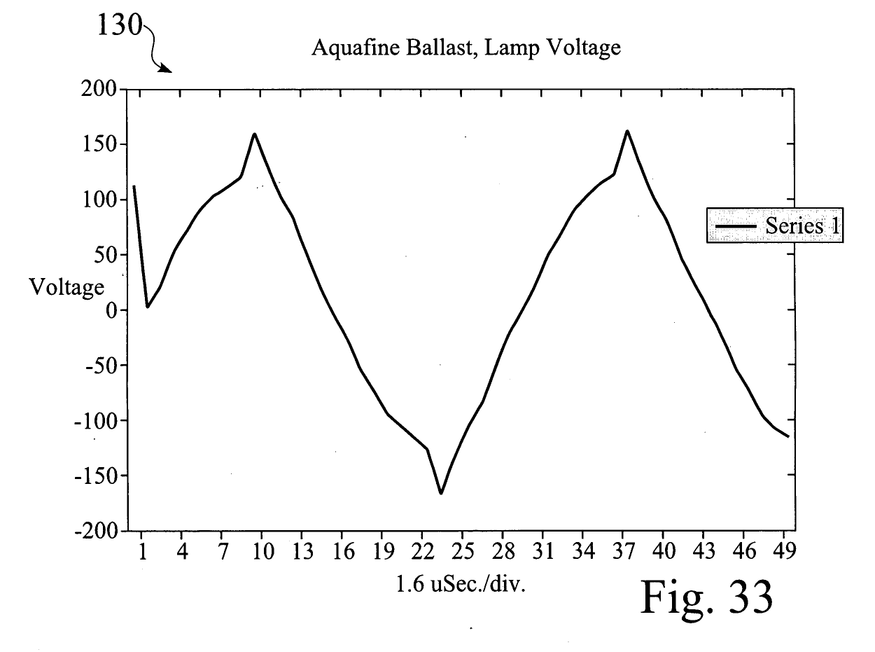

FIG. 33 furnishes a graph which shows how one embodiment of the lamp voltage for a ballast varies with time.

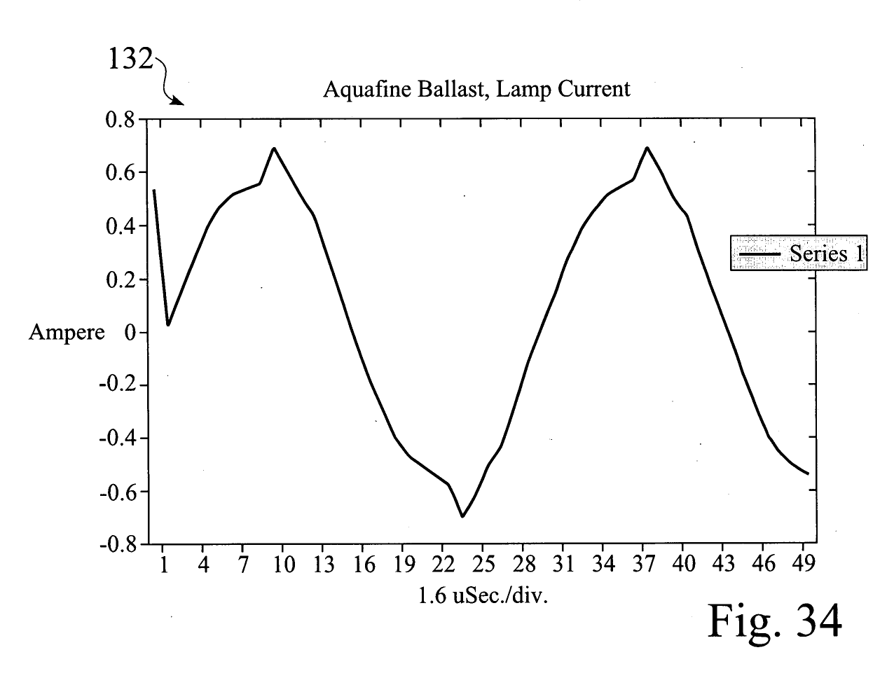

FIG. 34 supplies a graph which shows how one embodiment of the lamp current for a ballast varies with time.

In one embodiment of the invention, the first electrical signal which is applied across the electrodes of the lamp is a low voltage direct current. This first electrical signal stimulates the emission of photons from the ionized cloud within the sealed enclosure. The first electrical signal is applied for a specific first period of time.

During this first period of time, the ionization channel in the gas cloud within the lamp degrades. To bolster the ionization channel, a second electrical signal comprising a series of periodic pulses is applied to the lamp for a second period of time. In one embodiment, this second electrical signal has a higher voltage than the first electrical signal. By applying this combination of first and second signals, the photon production per total input power is higher than that which would be achieve using only the first electrical signal.

### CONCLUSION

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives that have been disclosed above are intended to educate the reader about preferred embodiments of the invention, and are not intended to constrain the limits of the invention or the scope of Claims.

### LIST OF REFERENCE CHARACTERS

10 Voltage Diagram
12 Excitation phase
14 Blending phase
16 Monitoring phase
18 Stabilization & Maintenance
20 Optional Reverse Polarity
22 Power supply circuit board
24 Power factor correction power supply
26 Microcontroller
28 Switchable filter
30 Switchable resistor
32 Electrical switch
34 Output relays
36 Lamp
37 Spectrum of light
38 UV sterilizer with dual bulbs
40 Outer UV transparent quartz jacket
42 Water
44 First bulb
46 Second bulb
48 First electrode
50 Second electrode
52 Embodiment using rectilinear bulb
54 Rectilinear bulb
56 Power source
58 Reflective layer
60 Rectilinear bulb embodiment with reflective coating
62 Reflective metal coating acts as electrical connection
64 UV sterilizer with multiple cylindrical bulbs
66 Inlet pipe
68 Outlet pipe
70 Cylindrical housing
72 Set of four UV bulbs
74 Rectilinear embodiment of UV sterilizer with multiple bulbs
78 Quartz envelope
80 Embodiment including rectilinear bulb with reflective coating
82 Rectilinear bulb with metal reflective coating
84 Reflective metal coating
86 Cross-sectional view of rectilinear bulb
86 View of ion formation
88 Quartz envelope
90 Switch
92 Ions
94 View of spiral electrodes
96 First spiral electrode
98 Second spiral electrode
100 End view of spiral electrodes
102 Enlarged view of one spiral electrode
104 Electrode protuberance
106 Enlarged view of group of protuberances
108 Enlarged view of one protuberance
110 Embodiment of bulb with sharp points
112 Sharp points
114 Battery
116 Alternative embodiment of bulb with sharp points
118 Graph of one embodiment of the first electrical signal which is applied across the electrodes of the lamp
120 Graph that shows one embodiment of the lamp current before an arc is initiated within the lamp
122 Graph of one embodiment of the first electrical signal after an arc is initiated within the lamp
124 View of one embodiment of current flowing through the lamp after an arc has been initiated
126 Graph of one embodiment of the combined first and second electrical signals employed by the present invention
128 View of lamp current, sub-hyper
130 Graph which shows how one embodiment of the lamp voltage for a ballast varies with time
132 Graph which shows how one embodiment of the lamp current for a ballast varies with time

What is claimed is:

1. A method comprising the steps of:
supplying a sealed enclosure for providing illumination;
said enclosure containing a plurality of molecules of a gas;
said enclosure having an interior surface; said interior surface being at least partially coated with a light emitting substance;
said enclosure including a first and a second electrode;
applying a first electrical signal across said first and said second electrodes to excite some of said plurality of molecules of a gas and to produce an ionized cloud within said enclosure; and
applying a second electrical signal across said first and said second electrodes along with said first electrical signal to maintain said ionized cloud within a set of predetermined limits to optimize the production of visible light from said light emitting substance on said interior surface of said enclosure.

2. A method as recited in claim 1, further comprising the steps of:
sensing the electrical impedance of said ionized cloud; and
varying said second electrical signal to optimize the production of visible light from said light emitting substance on said interior surface of said enclosure.

3. A method as recited in claim 1, further comprising the step of:
sensing an artifact; and
reversing the polarity of said second electrical signal to eliminate said artifact.

4. A method as recited in claim 1, in which:
said enclosure is formed from an optically transmissive substance.

5. A method as recited in claim 1, in which:
said enclosure is formed from glass.

6. A method as recited in claim 1, in which:
said enclosure is generally cylindrical.

7. A method as recited in claim 1, in which:
said enclosure is generally configured as a cylindrical spiral.

8. A method as recited in claim 1, in which:
said enclosure is a portion of a compact fluorescent bulb.

9. A method as recited in claim 1, in which:
said gas being selected to at least partially ionize when stimulated with electrical energy.

10. A method as recited in claim 1, in which:
said light emitting substance is fluorescent.

11. A method as recited in claim 1, in which:
said light emitting substance is phosphorescent.

12. A method as recited in claim 1, in which:
said first and said second electrodes being located generally at each end of said enclosure.

13. A method as recited in claim 1, in which:
said first and said electrodes are each connected to one pair of external electrodes.

14. A method as recited in claim 1, in which:
said first and said electrodes are each connected to a portion of a threaded conductive base that is configured to fit inside a conventional light bulb socket.

15. A method as recited in claim 1, in which:
some of said plurality of molecules of a gas become ionized when stimulated with electrical energy.

16. A method as recited in claim 1, in which:
said light emitting substance emits photons when some of said plurality of molecules of gas are ionized.

17. A method as recited in claim 1, in which:
said first electrical signal is a direct current.

18. A method as recited in claim 1, in which:
said first electrical signal is provided by a high impedance source.

19. A method as recited in claim 17, in which:
said direct current ranges between approximately 625 and 700 volts.

20. A method as recited in claim 1, in which:
said second electrical signal provides a mix of said direct current and an alternating current.

21. A method as recited in claim 1, in which:
said second electrical signal is provided by a low impedance source.

22. A method as recited in claim 20, in which:
said alternating current ranges approximately between 50 and 90 volts.

23. A method as recited in claim 1, in which:
said second electrical signal ranges approximately between 120 and 150 VDC.

24. A method as recited in claim 20, in which:
said alternating current has a frequency approximately between 65,000 and 90,000 cycles per second.

25. A method as recited in claim 1, in which:
said first electrical signal has a voltage range which depends upon the dimensions of said enclosure.

26. A method as recited in claim 1, in which:
said first electrical signal has a voltage range which depends upon the characteristics of said gas.

27. A method as recited in claim 1; in which:
said second electrical signal has a voltage range which depends upon the dimensions of said enclosure.

28. A method as recited in claim 1, in which:
said second electrical signal has a voltage range which depends upon the characteristics of said gas.

29. A method as recited in claim 1, in which:
said second electrical signal includes a plurality of high voltage refresh pulses.

30. A method as recited in claim 1, further comprising the step of:
installing a radio inside said enclosure.

31. A method as recited in claim 1, further comprising the step of:
attaching a radio to said enclosure.

32. A method as recited in claim 31, in which:
said radio is used to convey radio signals to help optimize the operation of a plurality of said enclosures.

33. A method as recited in claim 31, in which:
said radio is used to convey radio signals to furnish automatic dimming for a plurality of said enclosures.

34. A method as recited in claim 31, in which:
said radio operates in the Wi-Fi frequency band.

35. A method as recited in claim 31, in which:
said radio creates a Wi-Fi hotspot.

36. A method as recited in claim 1, further comprising the step of:
generating visible light using said enclosure without requiring an external ballast.

37. A method as recited in claim 31, in which:
said radio is also used for telecommunications.

38. A method as recited in claim 1, in which:
said interior surface of said enclosure also includes a partially mirrored surface to further enhance the optimization of the production of visible light from said light emitting substance on said interior surface of said enclosure.

39. A method as recited in claim 1, further comprising the step of:
using a priori knowledge of the characteristics of said enclosure allow for enhanced optimization of the production of visible light from said light emitting substance on said interior surface of said enclosure.

40. A method as recited in claim 32, in which:
said enclosure generates visible light; the intensity of said visible light being dimmable by adjusting said second electrical signal.

41. A method comprising the steps of:
confining a plasma;
stimulating said plasma with electrical energy to form a conductive plasma channel;
measuring the state of said conductive plasma channel by determining a mean impedance characteristic of said conductive plasma channel; and
managing said plasma channel to optimize its electrical impedance to provide efficient illumination.

42. An apparatus comprising:
a power factor correction power supply for supplying power;
a microcontroller connected to said power factor correction power supply for controlling said power factor correction power supply;
a switchable resistance; said switchable resistance connected to said switchable filter for varying the output impedance of said power factor correction power supply;
a switchable filter; said switchable filter connected to said power factor correction power supply for filtering the output of said electrical switch;
a lamp; said lamp connected to said output relay; said lamp for providing illumination;
an electrical switch; said electrical switch connected to said power factor correction power supply for controlling the operation of said lamp; and
an output relay; said output relay connected to said switchable filter for changing the polarity of energy applied to said lamp.

43. An apparatus comprising:
a power factor correction power supply means for providing power;
a microcontroller means connected to said power factor correction power supply for controlling said power factor correction power supply means;
a switchable filter means for enabling switchable filtering; said switchable filter means being connected to said power factor correction power supply means;
a switchable resistance means for providing a switchable resistance; said switchable resistance means being connected to said switchable filter means;
an output relay means for furnishing a controlled output; said output relay means connected to said switchable filter means;
a lamp means for providing illumination; said lamp means being connected to said output relay means; and
an electrical switch means for supplying on and off control; said electrical switch means connected to said lamp means.

44. A method comprising the steps of:
supplying a sealed enclosure for providing ultraviolet radiation for disinfection;
said enclosure containing a plurality of molecules of a gas;
said enclosure having an interior surface; said interior surface being at least partially coated with an ultraviolet radiation emitting substance;
said enclosure including a first and a second electrode;
applying a first electrical signal across said first and said second electrodes to excite some of said plurality of molecules of a gas and to produce an ionized cloud within said enclosure; and
applying a second electrical signal across said first and said second electrodes along with said first electrical signal to maintain said ionized cloud within a set of predetermined limits to optimize the production of said ultraviolet radiation from said ultraviolet radiation emitting substance on said interior surface of said enclosure.

45. A method as recited in claim 44, in which said ultraviolet radiation is used to disinfect water.

46. A method as recited in claim 44, in which said ultraviolet radiation is used to disinfect a liquid.

47. A method as recited in claim 44, in which said ultraviolet radiation is used to disinfect a gas.

48. A method as recited in claim 44, in which said ultraviolet radiation is used to disinfect a beverage.

49. A method as recited in claim 44, in which said ultraviolet radiation is used to disinfect food.

50. A method as recited in claim 44, in which said ultraviolet radiation is used to disinfect a surface.

51. A method as recited in claim 44, in which said ultraviolet radiation is used to disinfect an implement.

52. A method as recited in claim 44, comprising the additional steps of:
providing an additional one of said enclosures; and
operating said enclosures at less than full power to extend the useful lifetime of said enclosures.

53. A method as recited in claim 44, in which said enclosure is generally rectilinear.

54. A method as recited in claim 44, comprising the additional step of:
coating a portion of the inside of said enclosure with a reflective layer.

55. A method as recited in claim 54, in which said reflective layer is also used as a conductor for power for said enclosure 54.

56. A method as recited in claim 44, comprising the additional step of:
providing a plurality of enclosures for generating ultraviolet radiation for disinfection.

57. A method as recited in claim 44, in which said first and said second electrodes are configured in a generally cylindrical, spiral shape.

58. A method as recited in claim 54, in which said first and said second electrodes include a protuberance.

59. A method as recited in claim 44, in which said enclosure includes a sharp point.

60. A method comprising the steps of:
supplying a sealed enclosure for providing illumination;
said enclosure containing a plurality of molecules of a gas;
said enclosure having an interior surface; said interior surface being at least partially coated with a light emitting substance;
said enclosure including a first and a second electrode;
applying a first electrical signal across said first and said second electrodes to excite some of said plurality of molecules of a gas and to produce an ionization channel within said enclosure; and
applying a second electrical signal across said first and said second electrodes along with said first electrical signal to maintain said ionized cloud within a set of predetermined limits to optimize the production of visible light from said light emitting substance on said interior surface of said enclosure;
said first electrical signal being a low voltage direct current applied across said first and said second electrodes;
said first electrical signal being applied across said first and said second electrodes to stimulate the emission of photons from said ionized cloud within said sealed enclosure at high efficiency for a first period of time;
continuing to apply said first electrical signal during said first period of time;
applying said second electrical signal after the passage of said first period of time until said ionization channel in said cloud of gas degrades;
said second electrical signal being a plurality of periodic pulses; each of said plurality of periodic pulses being applied for a second period of time;
said second electrical signal having a higher voltage than said first electrical signal which maintains the effectiveness of said ionization channel within said sealed enclosure to provide higher photon production per total input power than would be provided using only said first electrical signal.
